(19) United States
(12) Patent Application Publication
(10) Pub. No.: US 2010/0256133 A1
(43) Pub. Date: Oct. 7, 2010

(54) NOVEL COMPOUNDS HAVING INDAZOLE FRAMEWORKS, METHODS FOR PREPARING THE SAME AND PHARMACEUTICAL COMPOSITION COMPRISING THE SAME

(76) Inventors: Jeom-Yong Kim, Seoul (KR); Mi-Ra Ma, Gyeonggi-do (KR); Kyung-Yun Jung, Gyeonggi-do (KR); Jin-Seok Choi, Gyeonggi-do (KR); Young-Seok Cho, Gyeonggi-do (KR); Sang-Hak Lee, Gyeonggi-do (KR)

Correspondence Address: DICKSTEIN SHAPIRO LLP, 1825 EYE STREET NW, Washington, DC, 20006-5403, US

(21) Appl. No.: 12/679,083

(22) PCT Filed: Sep. 19, 2008
(86) PCT No.: PCT/KR2008/005555

§ 371 Date: Mar. 19, 2010

(30) Foreign Application Priority Data
Sep. 21, 2007 (KR) 10-2007-0096984

(51) Int. Cl. A61K 31/437; C07D 471/04; A61K 31/5377; A61K 31/496; A61P 35/00; A61P 3/10; A61P 25/28; A61P 25/00

(52) U.S. Cl. 514/233.2; 546/119; 544/127; 544/362; 514/253.04; 514/303

(57) ABSTRACT

Novel compounds having indazole frameworks, as well as a method for preparing the same and a pharmaceutical composition comprising the same are provided. The compounds of the present invention can inhibit protein kinase activity and thus the pharmaceutical composition of the present invention can be used to prevent or treat diseases or disorders which are related to protein kinase activity.

### TECHNICAL FIELD

The present invention relates to novel compounds having an indazole framework, a preparation method thereof, and a pharmaceutical composition comprising the same. More particularly, the present invention relates to novel compounds or a pharmaceutically acceptable salt thereof, showing inhibitory activity against protein kinases and the composition may include, as an active ingredient, the compounds or the pharmaceutically acceptable salt thereof alone or in combination with other active ingredients. The compounds of the present invention are useful in the treatment of cancer, neurological diseases, autoimmune disease and other diseases which can be treated by a protein kinase inhibitor.

### BACKGROUND ART

Protein kinase is an enzyme that catalyzes the phosphorylation of specific residues within proteins, usually playing a fundamental role in signal transduction. This class of protein may further be separated into subsets such as one group which specifically phosphorylates residues of serine and/or threonine, another group which specifically phosphorylates residues of tyrosine, and yet another group which phosphorylates both of tyrosine and serine/threonine.

In fact, protein kinases are essential factors in signaling pathways responsible for the transduction of extracellular signals, including the nucleus-targeting actions of cytokines on their receptors, which cause various biological results. In normal cell physiology, protein kinases account for various roles including regulation of the cell cycle, cell growth, differentiation, apoptosis, cell mobility, and mitogenesis.

Protein kinases mediate intracellular signal transduction generally by effecting a phosphoryl transfer from a nucleoside triphosphate to a protein acceptor that is involved in a signaling pathway. This phosphorylation acts as a switch turning on or off target proteins or molecules, thus regulating or controlling the biological functions of target proteins. The phosphorylation is triggered ultimately in response to various extracellular and other stimuli.

Examples of such stimuli include environmental and chemical stress signals (e. g. osmotic shock, heat shock, UV radiation, bacterial endotoxins, H₂O₂), cytokines (e. g. interleukin-1 (IL-1)), tumor necrosis factor α (TNF-α), growth factors (e.g. granulocyte macrophage-colony-stimulating factor (GM-CSF)), and fibroblast growth factor. An extracellular stimulus may effect one or more cellular responses related to cell growth, migration, differentiation, secretion of hormones, activation of transcriptional factors, muscle contraction, glucose metabolism, control of protein synthesis, and regulation of cell cycle.

Meanwhile, kinase activity is implicated in the occurrence of various diseases. In fact, a number of diseases are associated with abnormal cellular responses triggered by protein kinase-mediated events. These diseases include autoimmune, inflammatory, metabolic, neurological, neurodegenerative and cardiovascular diseases, cancer, allergies, asthma, Alzheimer's disease, and hormone-related diseases. Accordingly, there has been a substantial effort in medicinal chemistry to find protein kinase inhibitors that are effective as therapeutic agents for kinase-associated diseases.

However, there is still a great need for new therapeutics for regulating such protein targets due to no or few alternatives of the currently available therapies for most of the pathologies associated with protein kinases.

Particularly important among the protein kinases is glycogen synthase kinase-3 (GSK-3), which is a serine/threonine protein kinase existing as two structurally similar isoforms α and β that are each encoded by distinct genes [see: Coghlan et al., Chemistry & Biology, 7, 793-803(2000); Kim and Kimmel, Curr. Opinion Genetics Dev., 10, 508-514(2000)]. GSK-3 is implicated in a wide array of disease processes including diabetes, Alzheimer's disease, CNS disorders (e.g., manic depressive disorder and neurodegenerative disease), and hypertrophic cardiomyopathy [see: PCT Publication Nos. WO99/65897; WO00/38675; Kaytor and Orr, Curr. Opin. Neurobiol., 12, 275-8 (2000); Haq et al., J. Cell Biol., 151, 117-30(2000); Eldar-Finkelman, Trends Mol. Med., 8, 126-32 (2002)]. These diseases may be caused by the abnormal operation of certain cell signaling pathways in which GSK-3 acts as a key regulator.

GSK-3 has been found to phosphorylate a number of regulatory proteins and thus modulate the activity thereof. These proteins include glycogen synthase, which is the rate-limiting enzyme necessary for glycogen synthesis, the microtubule-associated protein Tau, the gene transcription factor β-catenin, the translational initiation factor elF-2B, as well as ATP citrate lyase, axin, heat shock protein-1, c-Jun, c-myc, c-myb, CREB, and CEPBα. These diverse protein targets implicate GSK-3 in various biological aspects of cellular metabolism, proliferation, differentiation and development.

In a GSK-3 mediated pathway that is relevant for the treatment of type II diabetes, insulin-induced signaling leads to cellular glucose uptake and glycogen synthesis. Along this pathway, GSK-3 acts as a negative regulator of the insulin-induced signal. Normally, the presence of insulin causes inhibition of GSK-3 mediated phosphorylation and deactivation of glycogen synthase. The inhibition of GSK-3 leads to increased glycogen synthesis and glucose uptake [see: Klein et al., PNAS, 93, 8455-9 (1996); Cross et al., Biochem. J., 303, 21-26(1994); Cohen, Biochem. Soc. Trans., 21, 555-567(1993); Massillon et al., Biochem J. 299, 123-128 (1994); Cohen and Frame, Nat. Rev. Mol. Cell. Biol., 2, 769-76(2001)].

In a diabetic patient with impaired insulin response, however, glycogen synthesis and glucose uptake fail to increase in spite of the presence of relatively high blood levels of insulin. This leads to abnormally high blood levels of glucose with acute and long term effects that may ultimately result in cardiovascular disease, renal failure and blindness. In such patients, the normal insulin-induced inhibition of GSK-3 does not occur. It has also been reported that in patients with type II diabetes, GSK-3 is overexpressed [see: PCT Publication No. WO 00/38675]. Therefore, therapeutic inhibitors of GSK-3 may be useful in the treatment of diabetic patients suffering from an impaired response to insulin.

Also, GSK-3 is involved in myocardial infarction as disclosed in the following literature [see: Jonassen et al., Circ Res, 89:1191, 2001 (The reduction in myocardial infarction by insulin administration at reperfusion is mediated via Akt dependent signaling pathway); Matsui et al., Circulation, 104:330, 2001 (Akt activation preserves cardiac function and prevents cardiomyocyte injury after transient cardiac ischemia in vivo); Miao et al., J Mol Cell Cardiol, 32:2397, 2000 (Intracoronary, adenovirus-mediated Akt gene delivery in heart reduced gross infarct size following ischemia-reperfusion injury in vivo); and Fujio et al., Circulation et al., 101:660, 2000 (Akt signaling inhibits cardiac myocyte apoptosis in vitro and protects against ischemia-reperfusion injury in mouse heart)].

GSK-3 activity plays a role in head trauma as disclosed in the following literature [see: Noshita et al., Neurobiol Dis, 9:294, 2002 (Upregulation of Akt/PI3-kinase pathway may be crucial for cell survival after traumatic brain injury) and Dietrich et al., J Neurotrauma, 13:309, 1996 (Posttraumatic administration of bFGF significantly reduced damaged cortical neurons & total contusion volume in a rat model of traumatic brain injury)].

GSK-3 is also known to play a role in psychiatric disorders as disclosed in the following literature [see: Eldar-Finkelman, Trends Mol Med, 8:126, 2002; Li et al., Bipolar Disord, 4:137, 2002 (LiCl and Valproic acid, anti-psychotic and mood stabilizing drugs decrease GSK3 activities and increase beta-catenin) and Lijam et al., Cell, 90:895, 1997 (Dishevelled KO mice showed abnormal social behavior and defective sensorimotor gating. A dishevelled, cytoplamic protein involved in WNT pathway inhibits GSK3 beta activities)]. It has been shown that GSK3 inhibition by lithium and valproic acid induces axonal remodeling and change synaptic connectivity [see: Kaytor & Orr, Curr Opin Neurobiol, 12:275, 2002 (Downregulation of GSK3 causes changes in mirotubule-associated proteins: tau, MAP1 & 2) and Hall et al., Mol Cell Neurosci, 20:257, 2002 (Lithium and valproic acid induces the formation of growth cone-like structures along the axons)].

GSK-3 activity is also associated with Alzheimer's disease. This disease is characterized by the well-known β-amyloid peptide and the formation of intracellular neurofibrillary tangles. The neurofibrillary tangles contain hyperphosphorylated Tau protein where Tau is phosphorylated on abnormal sites. GSK-3 is shown to phosphorylate these abnormal sites in cell and animal models. Furthermore, the inhibition of GSK-3 has been shown to prevent hyperphosphorylation of Tau in cells [Lovestone et al., Current Biology 4, 1077-86 (1994); Brownlees et al., Neuroreport 8, 3251-55 (1997); Kaytor and Orr, Curr. Opin. Neurobiol., 12, 275-8(2000)]. Significant increased Tau hyperphosphorylation and abnormal morphology of neurons were observed in transgenic mice overexpressing GSK3 [Lucas et al., EMBO J, 20:27-39 (2001)]. Active GSK3 accumulates in the cytoplasm of pretangled neurons, which can lead to neurofibrillary tangles in brains of patients with AD [Pei et al., J Neuropathol Exp Neurol, 58, 1010-19 (1999)]. Therefore, if GSK-3 activity is inhibited, the generation of neurofibrillary tangles are slowed or halted, thus treating or reducing the severity of Alzheimer's disease.

Many in vitro evidence for the role that GSK-3 plays in Alzheimer's disease have now been obtained, as described in the following literature [see: Aplin et al (1996), J Neurochem 67:699; Sun et al (2002), Neurosci Lett 321:61 (GSK3b phosphorylates the cytoplasmic domain of Amyloid Precursor Protein (APP) and GSK3b inhibition reduces Ab40 & Ab42 secretion in APP-transfected cells); Takashima et al (1998), PNAS 95:9637; Kirschenbaum et al (2001), J Biol Chem 276:7366 (GSK3b complexes with and phosphorylates presenilin-1, which is associated with gamma-secretase activity in the synthesis of Aβ from APP); Takashima et al (1998), Neurosci Res 31:317 (Activation of GSK3b by Ab(25-35) enhances phosphorylation of tau in hippocampal neurons. This observation provides a link between Aβ and neurofibrillary tangles composed of hyperphosphorylated tau, another pathological hallmark of AD); Takashima et al (1993), PNAS 90:7789 (Blockade of GSK3b expression or activity prevents Ab-induced neuro-degeneration of cortical and hippocampal primary cultures); Suhara et al (2003), Neurobiol Aging. 24:437 (Intracellular Ab42 is toxic to endothelial cells by interfering with activation of Akt/GSK-3b signaling-dependent mechanism); De Ferrari et al (2003) Mol Psychiatry 8:195 (Lithium protects N2A cells & primary hippocampal neurons from Aβ fibrils-induced cytotoxicity, and reduced nuclear translocation/destabilization of b-catenin); and Pigino et al., J Neurosci, 23:4499, 2003 (The mutations in Alzheimer's presenilin 1 may deregulate and increase GSK-3 activity, which in turn, impairs axonal transport in neurons. The consequent reductions in axonal transport in affected neurons can ultimately lead to neurodegeneration)].

Evidence of the role that GSK-3 plays in Alzheimer's disease has been shown in vivo, as disclosed in the following literature [see: Yamaguchi et al (1996), Acta Neuropathol 92:232; Pei et al (1999), J Neuropath Exp Neurol 58:1010 (GSK3b immunoreactivity is elevated in susceptible regions of AD brains); Hernandez et al (2002), J Neurochem 83;1529 (Transgenic mice with conditional GSK3b overexpression exhibit cognitive deficits similar to those in transgenic APP mouse models of AD); De Ferrari et al (2003) Mol Psychiatry 8:195 (Chronic lithium treatment rescued neurodegeneration and behavioral impairments (Morris water maze) caused by intrahippocampal injection of Aβ fibrils,); McLaurin et al., Nature Med, 8: 1263, 2002 (Immunization with Aβ in a transgenic model of AD reduces both AD-like neuropathology and spatial memory impairments); and Phiel et al (2003) Nature 423:435 (GSK3 regulates amyloid-beta peptide production via direct inhibition of gamma secretase in AD tg mice)].

Presenilin-1 and kinesin-1 are also substrates for GSK-3 and relate to another mechanism for the role GSK-3 plays in Alzheimer's disease, as was recently described in the following literature [see: Pigino, G., et al., Journal of Neuroscience (23:4499, 2003)]. It was found that GSK3 beta phosphorylates a kinsesin-I light chain, which results in a release of kinesin-1 from membrane-bound organelles, leading to a reduction in fast anterograde axonal transport [see: Morfini et al., 2002].

The present inventors suggest that the mutations in PSI may deregulate and increase GSK-3 activity, which in turn, impairs axonal transport in neurons. The consequent reduction in axonal transport in affected neurons ultimately leads to neurodegeneration.

GSK-3 is also implicated in amyotrophic lateral sclerosis (ALS). Refer to the following literature [Williamson and Cleveland, 1999 (Axonal transport is retarded in a very early phase of ALS in mSOD1 mice); Morfini et al., 2002 (GSK3 phosphorylates kinesin light chains and inhibits anterograde axonal transport); Warita et al., Apoptosis, 6:345, 2001 (The majority of spinal motor neurons lost the immunoreactivities for both PI3-K and Akt in the early and presymptomatic stage that preceded significant loss of the neurons in this SOD1 tg animal model of ALS); and Sanchez et al., 2001 (The inhibition of PI-3K induces neurite retraction mediated by GSK3 activation)]. GSK-3 activity is also linked to spinal cord and peripheral nerve injuries. Refer to the following literature [Grothe et al., Brain Res, 885:172, 2000 (FGF2 stimulate Schwann cell proliferation and inhibit myelination during axonal growth); Grothe and Nikkhah, 2001 (FGF-2 is up regulated in the proximal and distal nerve stumps within 5 hours after nerve crush); and Sanchez et al., 2001 (The inhibition of PI-3K induces neurite retraction mediated by GSK3 activation)].

Another substrate of GSK-3 is β-catenin, which is degraded after phosphorylation by GSK-3. Reduced levels of β-catenin have been reported in schizophrenic patients and have also been associated with other diseases related to an increase in neuronal cell death [see: Zhong et al., Nature, 395, 698-702 (1998); Takashima et al., PNAS, 90, 7789-93 (1993); Pei et al., J. Neuropathol. Exp, 56, 70-78 (1997); and Smith et al., Bio-org. Med. Chem. 11, 635-639 (2001)]. Further, β-catenin and Tcf-4 play a dual role in vascular remodeling by inhibiting vascular smooth muscle cell apoptosis and promoting proliferation [see: Wang et al., Circ Res, 90:340, 2002). Accordingly, GSK-3 is related to angiogenic impairments. Refer to the following literature [Liu et al., FASEB J, 16:950, 2002 (Activation of GSK3 reduces hepatocyte growth factor, causing altered endothelial cell barrier function and diminished vascular integrity) and Kim et al., k J Biol Chem, 277:41888, 2002 (GSK3beta activation inhibits angiogenesis in vivo using Matrigel plug assay: the inhibition of GSK3beta signaling enhances capillary formation)].

Relationships between GSK-3 and Huntington's disease has been shown. Refer to the following literature [Carmichael et al., J Biol Chem., 277:33791, 2002 (GSK3 beta inhibition protects cells from poly-glutamine-induced neuronal and non-neuronal cell death via increases in b-catenin and its associated transcriptional pathway)]. Overexpression of GSK3 reduces the activation of heat shock transcription factor-1 and heat shock protein HSP70 [see: Bijur et al., J Biol Chem, 275:7583, 2000] that are shown to decrease both poly-(Q) aggregates and cell death in in vitro HD model [see:Wyttenbach et a ., Hum Mol Genet, 11:1137, 2002].

GSK-3 effects the levels of FGF-2 and their receptors are increased during remyelination of brain aggregate cultures remyelinating rat brains. Refer to the following literature [Copelman et al., 2000, Messersmith, et al., 2000; and Hinks and Franklin, 2000]. It was also found that FGF-2 induces process outgrowth by oligodendrocytes implicating the involvement of FGF in remyelination [see: Oh and Yong, 1996; Gogate et al., 1994] and that FGF-2 gene therapy has shown to improve the recovery of experimental allergic encephalomyelitis (EAE) mice [see: Ruffini, et al., 2001].

Association between GSK-3 and hair growth was found because Wnt/beta-catenin signaling is shown to play a major role in hair follicle morphogenesis and differentiation [see: Kishimotot et al, Genes Dev, 14:1181, 2000; Millar, J Invest Dermatol, 118:216, 2002]. It was found that mice with constitutive overexpression of the inhibitors of Wnt signaling in skin failed to develop hair follicles. Wnt signals are required for the initial development of hair follicles and GSK3 constitutively regulates Wnt pathways by inhibiting beta-catenin [see: Andl et al., Dev Cell 2:643, 2002]. A transient Wnt signal provides the crucial initial stimulus for the start of a new hair growth cycle, by activating beta-catenin and TCF-regulated gene transcription in epithelial hair follicle precursors [see: Van Mater et al., Genes Dev, 17:1219, 2003].

Because GSK-3 activity is associated with sperm motility, GSK-3 inhibition is useful as a male contraceptive. It was shown that a decline in sperm GSK3 activity is associated with sperm motility development in bovine and monkey epididymis [see: Vijayaraghavan et al., Biol Reprod, 54: 709, 1996; Smith et al., J Androl, 20:47, 1999]. Furthermore, tyrosine and serine/threonine phosphorylation of GSK3 is high in motility compared to immotile sperm in bulls [see: Vijayaraghavan et al., Biol Reprod, 62:1647, 2000]. This effect was also demonstrated with human sperm [see: Luconi et al., Human Reprod, 16:1931, 2001].

As a consequence of the biochemical importance of protein kinases, keen attention is now paid to protein kinase inhibitors which are therapeutically useful. Accordingly, there is still a need for the development of protein kinase inhibitors that are useful in the treatment of various diseases or conditions associated with protein kinase activation.

### DISCLOSURE OF INVENTION

### Technical Problem

It is therefore an object of the present invention to provide a novel compound which may be used as a protein kinase inhibitor which is therapeutically useful, a method for the preparation thereof, and a pharmaceutical composition comprising the same as an active ingredient. The present invention provides a novel compound which can serve as a protein kinase inhibitor, as well as being useful in the treatment of various diseases or conditions associated with the activation of protein kinases, and a method for preparing the same.

### Technical Solution

The present invention provides a novel compound which can serve as a protein kinase inhibitor useful in the treatment of various diseases or conditions associated with the activation of protein kinases, and which can be easily prepared, and a method for preparing the same.

Particularly, the present invention provides a novel compound, represented by the following Chemical Formula 1, or a pharmaceutically acceptable salt thereof.

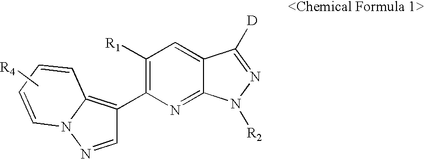

wherein,

D is hydrogen or —NR₃R₃′,

R₁ is hydrogen, a straight or branched C₁˜C₈ alkyl, a C₁˜C₈ alkoxy, or halogen,

R₂, R₃, and R₃′ are each independently hydrogen, a straight or branched C₁˜C₈ alkyl, or —(X₁)—R₅,

wherein,
R₃ and R₃′ may be combined to each other to form a 6-membered heterocycloalkyl, which is unsubstituted or substituted with a straight or branched C₁˜C₈ alkyl and contains one or more heteroatoms selected from N and O,

X₁ is a straight or branched C₁˜C₈ alkylene, —O—, —CO—, —(CO)₂—, —(SO)—, —(SO₂)—, —CH₂(C═O)—, —C(═O)CH₂—, or a single bond; and
R₅ is hydroxy; carboxy; a straight or branched C₁˜C₈ alkyl; a straight or branched C₁˜C₈ alkyl substituted with a C₂˜C₈ dialkylamino; a straight or branched C₁˜C₈ alkyl substituted with a C₆˜C₂₀ aryl; a straight or branched C₁˜C₈ alkyl substituted with a halogen-substituted C₆˜C₂₀ aryl; a straight or branched C₁˜C₈ alkyl substituted with a C₃˜C₈ heterocycloalkyl containing one or more heteroatoms selected from N and O; a straight or branched C₁˜C₈ alkyl substituted with a C₃˜C₈ heterocycloalkyl which is substituted with a straight or branched C₁˜C₈ alkyl and contains one or more heteroatoms selected from N and O; a C₃˜C₈ cycloalkyl; a straight or branched C₁˜C₈ hydroxyalkyl; a C₁˜C₈ alkoxy; a C₁˜C₈ acetoxy; a C₂˜C₈ alkenyl; nitryl; a C₂˜C₈ alkenyl substituted with a C₆˜C₂₀ aryl; a C₂˜C₈ alkenyl substituted with a halogen-substituted C₆˜C₂₀ aryl; a C₂˜C₈ alkynyl; a C₂˜C₈ alkynyl substituted with a C₆˜C₂₀ aryl; a C₂˜C₈ alkynyl substituted with a halogen-substituted C₆˜C₂₀ aryl; a C₆˜C₂₀ aryl; a C₆˜C₂₀ aryl substituted with one or more substituents selected from halogen, cyano, a straight or branched C₁˜C₈ alkyl, CF₃, amino, SO₂ and a C₁˜C₈ alkoxy; a C₆˜C₂₀ aryl substituted with a C₃˜C₈ heterocycloalkyl containing one or more heteroatoms selected from N and O; a C₆˜C₂₀ aryl substituted with a C₃˜C₈ heterocycloalkyl which is substituted with a straight or branched C₁˜C₈ alkyl and contains one or more heteroatoms selected from N and O; a C₆˜C₂₀ aryl substituted with a straight or branched C₁˜C₈ alkyl substituted with a C₃˜C₈ heterocycloalkyl containing one or more heteroatoms selected from N and O; a C₆˜C₂₀ aryl substituted with a straight or branched C₁˜C₈ alkyl substituted with a C₃˜C₈ hetrocycloalkyl which is substituted with a straight or branched C₁˜C₈alkyl and contains one or more heteroatoms selected from N and O; a C₆˜C₂₀ aryl substituted with a C₂˜C₈ dialkylamino; a C₃˜C₈ heterocycloalkyl containing one or more heteroatoms selected from N and O; a C₃˜C₈ heterocycloalkyl which is unsubstituted or substituted with halogen and contains one or more heteroatoms selected from N and O; phosphonate; phosphonate which is unsubsitituted or substituted with a straight or branched C₁˜C₈ alkyl; or NA₁A₂,
wherein,
   A₁ or A₂ may be the same or different and are each independently hydrogen; a straight or branched C₁˜C₈ alkyl; a straight or branched C₁˜C₈ alkyl which is unsubstituted or substituted with phenyl; a C₂˜C₈ alkenyl; a C₆˜C₂₀ aryl; a halogen-substituted C₆˜C₂₀ aryl; a C₆˜C₂₀ aryl substituted with a straight or branched C₁˜C₄ alkyl; or a C₆˜C₂₀ aryl substituted with a C₁˜C₄ alkoxy,

R₄ is hydrogen, hydroxy, halogen, a C₂˜C₈ dialkylamino, or —(X₂)—R₆;

wherein,
X₂ is a straight or branched C₁˜C₈ alkylene, a C₂˜C₈ alkenylene, a C₆˜C₂₀ arylene, a single bond, CO or SO₂, and
R₆ is a straight or branched C₁˜C₈ alkyl; a straight or branched C₁˜C₈ alkyl substituted with a C₆˜C₂₀ aryl; a straight or branched C₁˜C₈ alkyl substituted with a halogen-substituted C₆˜C₂₀ aryl; a C₃˜C₈ cycloalkyl; a C₁˜C₈ alkoxy; a C₂˜C₈ alkenyl; a C₂˜C₈ alkenyl substituted with a C₆˜C₂₀ aryl; a C₂˜C₈ alkenyl substituted with a C₆˜C₂₀ aryl substituted with a C₁˜C₈ alkoxy; a C₂˜C₈ alkenyl substituted with a halogen-substituted C₆˜C₂₀ aryl; a C₂˜C₈ alkynyl; a C₂˜C₈ alkynyl substituted with a C₆˜C₂₀ aryl; a C₂˜C₈ alkynyl substituted with a halogen-substituted C₆˜C₂₀ aryl; a C₆˜C₂₀ aryl; a C₆˜C₂₀ aryl substituted with a C₁˜C₈ alkoxy; a C₆˜C₂₀ aryl substituted with a straight or branched C₁˜C₈ alkyl; a halogen-substituted C₆˜C₂₀ aryl; a C₆˜C₂₀ aryl substituted with a halogen-substituted, straight or branched C₁˜C₈ alkyl; or a C₃˜C₈ heterocycloalkyl containing one or more heteroatoms selected from N and O.

In the C₃˜C₈ heterocycloalkyl containing one or more heteroatoms selected from N and O, the number of heteroatoms is preferably one or two.

More preferably, in the compound of Chemical Formula 1,

R₁ is a fluorine atom,

R₂, R₃, and R₃′ are each independently hydrogen or —(X₁)—R₅,
wherein,
X₁ is a straight or branched C₁˜C₈ alkylene, —CH₂(C═O)—, —C(═O)CH₂—, a single bond, —O— or —CO—, and

R₅ is a straight or branched C₁˜C₈ alkyl; a straight or branched C₁˜C₈ alkyl substituted with a C₂˜C₈ dialkylamino; a straight or branched C₁˜C₈ alkyl substituted with a C₆˜C₂₀ aryl; a straight or branched C₁˜C₈ alkyl substituted with a halogen-substituted C₆˜C₂₀ aryl; a straight or branched C₁˜C₈ alkyl substituted with a C₃˜C₈ heterocycloalkyl containing one or more heteroatoms selected from N and O; a straight or branched C₁˜C₈ alkyl substituted with a C₃˜C₈ heterocycloalkyl which is substituted with a straight or branched C₁˜C₈ alkyl and contains one or more heteroatoms selected from N and O; a C₃˜C₈ cycloalkyl; a straight or branched C₁˜C₈ alkanol; a C₁˜C₈ alkoxy; a C₂˜C₈ alkenyl; a C₂˜C₈ alkynyl; hydroxy; carboxy; a C₆˜C₂₀ aryl; a C₁˜C₈ acetoxy; a C₆˜C₂₀ aryl substituted with one or more substituents selected from halogen, cyano, a straight or branched C₁˜C₈ alkyl, CF₃ and a C₁˜C₈ alkoxy; a C₆˜C₂₀ aryl substituted with a C₃˜C₈ heterocycloalkyl containing one or more heteroatoms selected from N and O; a C₆˜C₂₀ aryl substituted with a C₃˜C₈ heterocycloalkyl which is substituted with a straight or branched C₁˜C₈ alkyl and contains one or more heteroatoms selected from N and O; a C₆˜C₂₀ aryl substituted with a straight or branched C₁˜C₈ alkyl substituted with a C₃˜C₈ heterocycloalkyl containing one or more heteroatoms selected from N and O; a C₆˜C₂₀ aryl substituted with a straight or branched C₁˜C₈ alkyl substituted with a C₃˜C₈ hetetocycloalkyl which is unsubstituted or substituted with a straight or branched C₁˜C₈ alkyl and contains one or more heteroatoms selected from N and O; a C₆˜C₂₀ aryl substituted with a C₂˜C₈ dialkylamino; a C₃˜C₈ heterocycloalkyl containing one or more heteroatoms selected from N and O; a halogen-substituted C₃˜C₈ heterocycloalkyl containing one or more heteroatoms selected from N and O; phosphonate; phosphonate which is substituted with a straight or branched C₁˜C₈ alkyl; nitryl; or a C₁˜C₈ alkylamino,

R₄ is hydrogen, hydroxy, halogen, a C₂˜C₈ dialkylamino or —(X₂)—R₆;
wherein
X₂ is a C₆˜C₂₀ arylene, CO, a single bond or SO₂, and
R₆ is a straight or branched C₁˜C₈ alkyl; a straight or branched C₁˜C₈ alkyl substituted with a halogen-substituted C₆˜C₂₀ aryl; a C₁˜C₈ alkoxy; a C₆˜C₂₀ aryl substituted with a C₁˜C₈ alkoxy; a halogen-substituted C₆˜C₂₀ aryl; or a C₆˜C₂₀ aryl substituted with a halogen-substituted straight or branched C₁˜C₈ alkyl.

Also, the present invention provides a method for preparing the above compound and a pharmaceutical composition for the treatment or prophylaxis of a disease or condition associated with protein kinase activity.

### Advantageous Effects

Being inhibitory of protein kinases, the novel compounds and the salts thereof in accordance with the present invention may be therapeutically useful in treating various diseases associated with the activation of protein kinases. Particularly, the novel compounds or salts thereof can be used for the treatment or prevention of cancer, diabetes, Alzheimer's disease, CNS disorders, and hypertrophic cardiomyopathy.

### MODE FOR INVENTION

The present invention pertains to a novel compound, represented by the following Chemical Formula 1, having inhibitory activity against protein kinases, a method for the preparation thereof, and a pharmaceutical composition comprising the same as an active ingredient.

Below, a detailed description will be given of the present invention.

1. Novel Compounds

The compound according to the present invention is a compound represented by the following Chemical Formula 1 and a salt thereof:

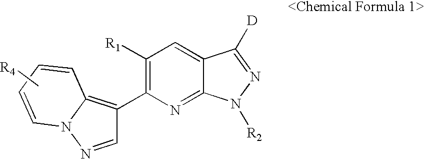

wherein D, R₁, R₂, and R₄ are as defined as above.

The pharmaceutically acceptable salts of the compound of Chemical Formula 1 in accordance with the present invention may be inorganic salts such as those of hydrochloric acid, bromic acid, sulfuric acid, and phosphoric acid or organic salts thereof such as citrate, acetate, lactate, tartarate, furmarate, formate, propionate, oxalate, trifluoroacetate, methanesulfonate, maleic aicd benzoate, gluconate, glyconate, succinate, 4-toluenesulfonate, galacturonate, embonate, glutamate and aspartate.

2. Preparation Methods of Novel Compounds

The novel compound of the present invention, represented by Chemical Formula 1, may be prepared according to the following Reaction Scheme 1.

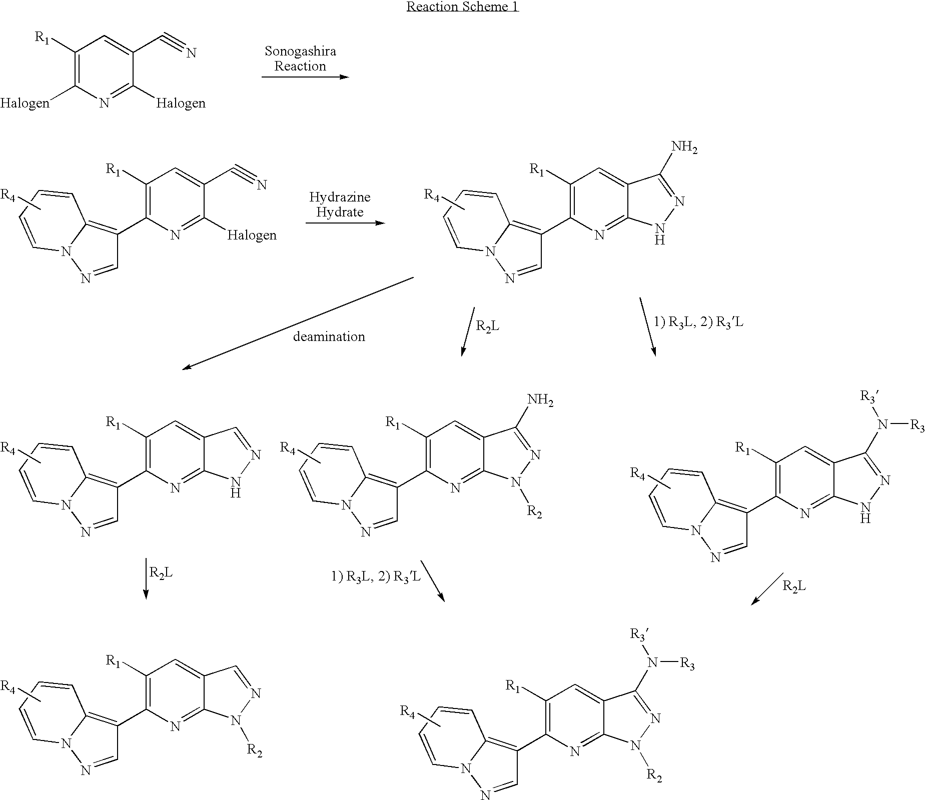

A compound represented by the following Chemical Formula 2 among the novel compounds represented by Chemical Formula 1 may be prepared according to the following Reaction Scheme 2;

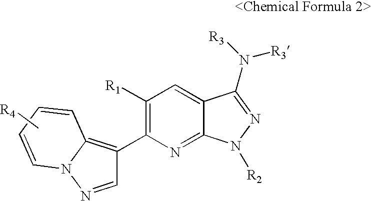

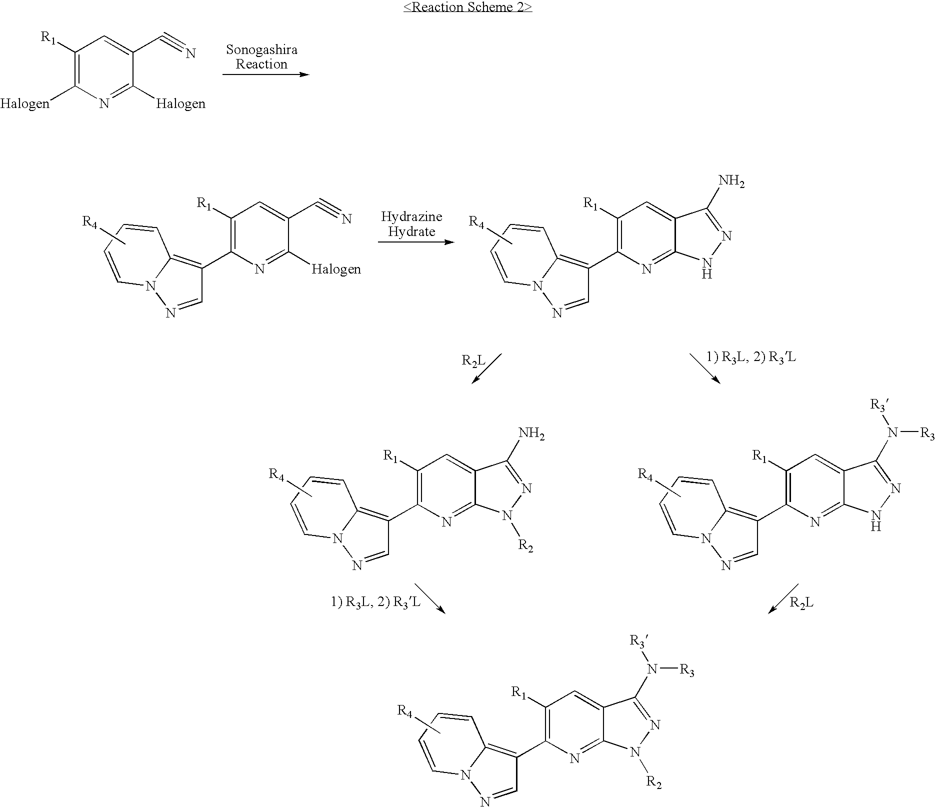

With reference to Reaction Scheme 2, the compounds of Chemical Formula 2 are prepared by:

Subjecting a compound of Chemical Formula 4 to the sonogashira reaction to obtain a compound of the following Chemical Formula 5;

Reacting the compound of Chemical Formula 5 with a hydrazine compound to obtain a compound of Chemical Formula 6; and

Reacting the compound of Chemical Formula 6 with a compound represented by R₂L or with compounds represented by R₃L and R₃′L.

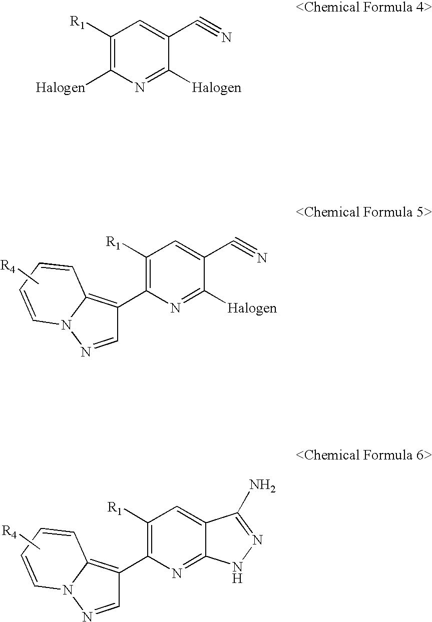

wherein, R₁, R₂, R₃, R₃′ and R₄ are as defined as in Chemical Formula 1, and in R₂L, R₃L and R₃′L, L is a leaving group. Examples of the leaving group include halogen and alcohol groups, but are not limited thereto.

The compounds according to the present invention may be prepared using any of typical methods. The synthesis thereof is suggested in the following Preparation Examples.

For example, the compound of Chemical Formula 2 may be prepared by following the reaction routes explained in the following Reaction Scheme 3.

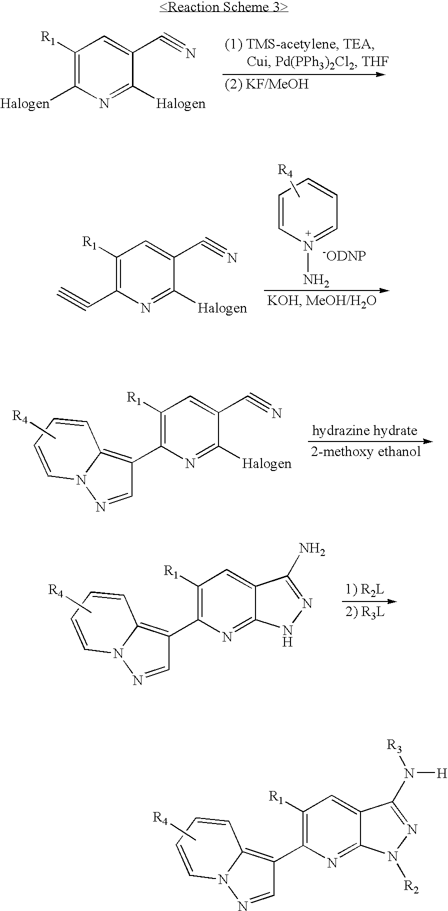

wherein, R₁, R₂, R₃, and R₄ are as defined as in Chemical Formula 1, and in R₂L and R₃L, L is a leaving group. Typically, the leaving group may include halogen and alcohol groups, but is not limited thereto.

A compound represented by the following Chemical Formula 3, falling within the range of the compound of Chemical Formula 1, may be prepared according to the following Reaction Scheme 4.

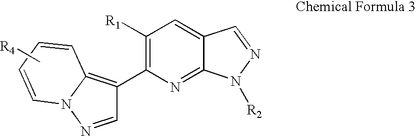

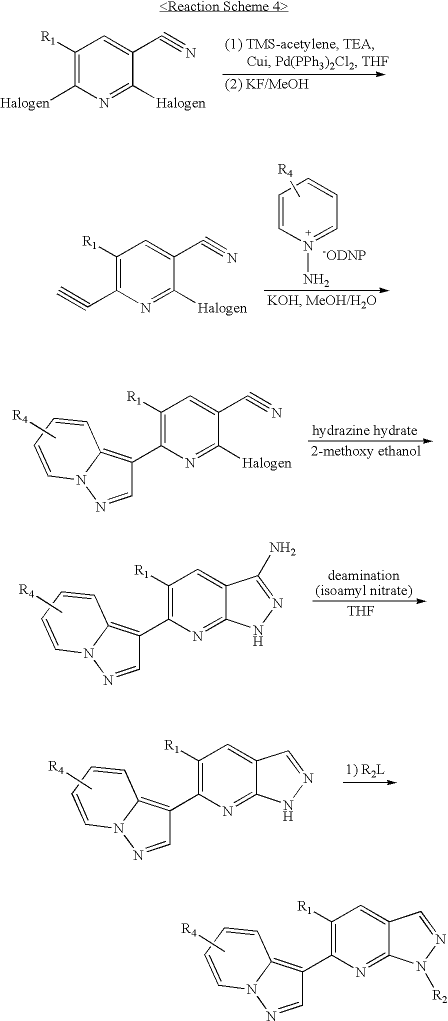

As illustrated in Reaction Scheme 4, the compound of Chemical Formula 3 may be prepared by:

Subjecting a compound of Chemical Formula 4 to a sonogashira reaction to obtain a compound of the following Chemical Formula 5;

Reacting the compound of the following Chemical Formula 5 with a hydrazine compound to obtain a compound of the following Chemical Formula 6;

Deaminating the compound of the following Chemical Formula 6 to a compound of the following Chemical Formula 7; and

Reacting the compound of the following Chemical Formula 7 with a compound represented by R₂L.

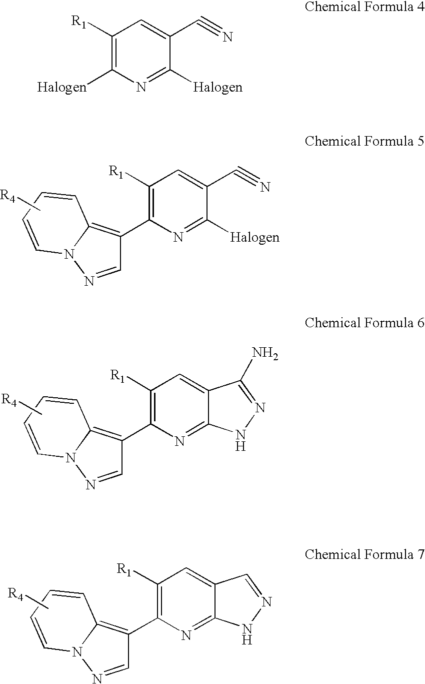

wherein, R₁, R₂, and R₄ are as defined as in Chemical Formula 1, and in R₂L, L is a leaving group. Examples of the leaving group typically include a halogen and alcohol groups, but are not limited thereto.

The following pyridinium imide derivative is a material used in the second step of Reaction Scheme 2 and can be synthesized as described below.

I. Synthesis of Pyridinium Imide Derivative

### Synthesis Example 1

### Preparation of 2-(2,4-dinitro-phenoxy)-isoindol-1,3(2H)-dione

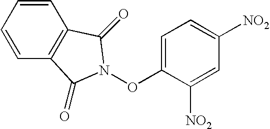

To a suspension of N-hydroxyphthalimide (25.0 g, 0.153 mol) in 500 ml of acetone was added triethylamine (21.5 ml, 0.154 mol). Then, the mixture was stirred and became dark red in color and the N-hydroxyphthalimide was slowly dissolved. The stirring was continued (about 10 min) until the mixture became homogeneous. After the addition of 2,4-dinitrochlorobenzene (31 g, 0.153 mol), the reaction mixture was further stirred for an additional 2 hours and turned into a light yellow suspension. It was poured into cold water (500 ml) to give precipitates which were subsequently filtered. The filtrate thus obtained was washed three times with cold MeOH and then three times with 100 ml of hexane to give the desired product as a white solid (48 g, yield 98%).

¹H-NMR (300 MHz, CDCl₃): δ 7.43 (d, J=9.3 Hz, 1H), 7.88-7.91(m, 2H), 7.96-7.99(m, 2H), 8.41(dd, J=2.5 Hz, J=9.3 Hz, 1H), 8.97(d, J=2.7 Hz, 1H)

### Synthesis Example 2

### Preparation of O-(2,4-Dinitrophenyl)hydroxyamine

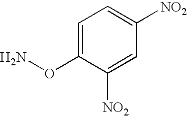

To a solution of 2-(2,4-dinitro-phenoxy)-isoindol-1,3(2H)-dione (20 g, 60.7 mmol) in CH₂Cl₂ was added hydrazine hydrate (8.86 ml, 0.18 mmol) in MeOH at 0° C. The reaction mixture quickly turned light yellow with the concomitant production of precipitates. After being left at 0° C. for 30 min, the suspension was mixed with cold aqueous HCl (1N, 400 ml) and agitated at 0° C. at high speed. The reaction mixture was filtered through a Buchner funnel and the precipitate was washed three times with ACN (50 ml). The filtrate was poured into a separatory funnel to separate the organic phase while the aqueous solution was extracted using CH₂Cl₂. The resulting organic phases were pooled, dehydrated over Na₂SO₄, filtered and concentrated in vacuo to give the desired product as an organic solid (12 g, yield 83%).

¹H-NMR (300 MHz, CDCl₃): 6.35 (brs, 2H), 7.99 (d, J=9.4 Hz, 1H), 8.37 (dd, J=2.7 Hz, J=9.4 Hz, 1H), 8.76(d, J=2.8 Hz, 1H)

### Synthesis Example 3

### Preparation of 2,4-Dinitro-phenolate 1-amino-4-methoxy-pyridinium

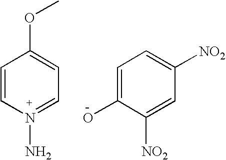

4-Methoxy pyridine (3.8 ml, 0.037 mmol) and O-(2,4-dinitrophenyl)hydroxylamine (8.19 g, 0.041 mmol) were mixed in ACN. After sealing the reaction vessel, the reaction mixture was stirred at 40° C. for 24 hours, and then concentrated. The residue thus obtained was pulverized using Et₂O, filtered, and dried in vacuo to give 2,4-dinitro-phenolate 1-amino-4-methoxy-pyridinium as a bright orange solid (11 g, yield 95%).

¹H-NMR (300 MHz, DMSO-d₆): 4.04(s, 3H), 6.31(d, J=9.7 Hz, 1H), 7.51(d, J=7.4 Hz, 2H), 7.75-7.81(m, 3H), 8.58(d, J=3.2 Hz, 1H), 8.65(d, J=7.5 Hz, 2H)

### Synthesis Example 4

### Preparation of 2,4-Dinitro-phenolate 1-amino-2-methyl-pyridinium

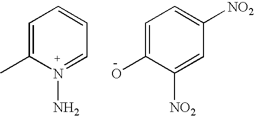

The same procedure as in Synthesis Example 3 was repeated, with the exception that 2-methyl pyridine was used as a starting material, to give the title compound as a bright orange solid.

¹H-NMR (300 MHz, DMSO-d₆): 2.71(s, 3H), 6.29 (d, J=9.9 Hz, 1H), 7.75 (dd, J=2.9 Hz, J=9.6 Hz, 1H), 7.86 (dd, J=7.4 Hz, 1H), 7.95 (d, J=7.6 Hz, 1H), 8.03 (brs, 2H), 8.20 (dd, =7.8 Hz, 1H), 8.58(d, J=2.9 Hz, 1H), 8.78 (d, J=6.3 Hz, 1H)

II . Synthesis of Pyrazole-Pyridine Derivative

### Synthesis Example 5

### Preparation of 2-Chloro-6-ethynyl-5-fluoro-nicotinonitrile

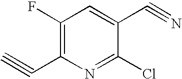

To a solution of 2,6-dichloro-5-fluoro-3-pyridinecarbonitrile (20 g, 0.105 mol) were added triethylamine (29 ml, 0.209 mol), copper iodide (1.99 g, 0.01 mol) and palladium chloride bis-triphenyl phosphine (3.67 g, 0.005 mol). To this reaction mixture was slowly added TMS-acetylene (17.4 ml, 0.12 mol), and the reaction mixture was stirred at room temperature for overnight. The reaction mixture was diluted with hexane and the solid were filtered off. To the crude silylated intermediate in MeOH was added potassium fluoride (6.08 g, 0.104 mol) and the mixture was stirred at room temperature for 10 min. Concentration under vacuum followed by column chromatography over silica gel with ethylacetate/hexane gave 7.6 g of solid (yield 40%).

¹H-NMR (300 MHz, CDCl₃): 3.70(s, 1H), 7.72(d, J=7 Hz, 1H)

### Synthesis Example 6

### Preparation of 2-Chloro-5-fluoro-6-pyrazole[1,5-a]pyridin-3-yl-nicotinonitrile Derivative

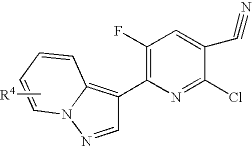

wherein R₄ is as defined as in Chemical Formula 1.

To a stirred solution of 2-chloro-6-ethynyl-5-fluoro-nicotinonitrile (0.011 mol) in THF were added a pyridinium derivative (0.013 mol) and K₂CO₃ (0.033 mol). This reaction mixture was stirred overnight at room temperature and evaporated under vacuum to remove the solvent. Following the addition of MC (methylene chloride), the reaction mixture was washed with water. The organic phase was dehydrated over Na₂SO₄ and concentrated under vacuum. Column chromatography using ethyl acetate/hexane afforded the desired product.

### Synthesis Example 7

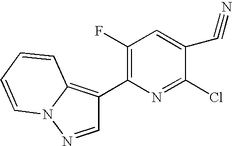

The title compound was prepared as a bright orange solid in the same manner as in Synthesis Example 6, with the exception that 2,4-dinitro-phenolate 1-amino-4-methoxy-pyridinium was used as a starting material (yield 50%).

¹H-NMR (300 MHz, CDCl₃): 7.03(dd, J=6.99 Hz, 1H), 7.48(dd, J=7 Hz, 1H), 7.66(d, J=10 Hz, 1H), 8.58-8.63(m, 2H), 8.67(d, J=9 Hz, 1H)

### Synthesis Example 8

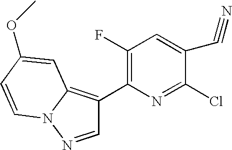

The title compound was prepared as a bright orange solid in the same manner as in Synthesis Example 6, with the exception that the compound synthesized in Synthesis Example 3 was used as a starting material (yield 35%).

¹H-NMR (300 MHz, CDCl₃): 3.98(s, 3H), 6.69(dd, J=2.9 Hz, J=7.6 Hz, 1H), 7.60(d, J=10.1 Hz, 1H), 8.02(d, J=2.7 Hz, 1H), 8.37(d, J=7.6 Hz, 1H), 8.51(d, J=3.9 Hz, 1H)

### Synthesis Example 9

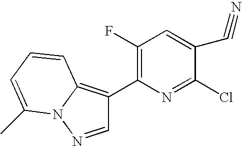

The title compound was prepared as a bright orange solid in the same manner as in Synthesis Example 6, with the exception that the compound synthesized in Synthesis Example 4 was used as a starting material (yield 35%).

¹H-NMR (300 MHz, CDCl₃): 2.83(s, 3H), 6.92(d, J=7 Hz, 1H), 7.42(dd, J=8.9 Hz, 1H), 7.63(d, J=10 Hz, 1H), 8.58(d, J=8.9 Hz, 1H), 8.63(d, J=3.9 Hz, 1H)

### Synthesis Example 10

### Preparation of 5-Fluoro-6-pyrazole[1,5-a]pyridin-3-yl-1H-pyrazolo[3,4-b]pyridin-3-yl-amine Derivative

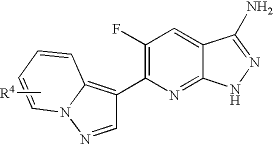

wherein R₄ is as defined as in Chemical Formula 1.

To a stirred solution of 2-chloro-5-fluoro-6-pyrazole[1,5-a]pyridin-3-yl-nicotinonitrile derivative (0.01 mol) in 2-methoxy ethanol was added hydrazine hydrate (0.05 mol), followed by refluxing overnight. The residue resulting from the evaporation of the solvent was pulverized using Et₂O, filtered, and dehydrated in vacuo to afford the desired product as a yellow solid.

### Synthesis Example 11

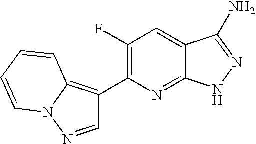

The same procedure as in Synthesis Example 10 was repeated, with the exception that the compound synthesized in Synthesis Example 7 was used as a starting material, to afford the title compound as a bright orange solid.

¹H-NMR (300 MHz, DMSO-d6): 5.53(brs, 2H), 7.11(dd, J=6.9 Hz, J=8.9 Hz, 1H), 7.52(dd, J=6.4 Hz, J=6.8 Hz, 1H), 8.02(d, J=11.8 Hz, 1H), 8.55(d, J=4.2 Hz, 1H), 8.68(d, J=9 Hz, 1H), 8.82(d, J=7 Hz, 1H), 11.98(brs, 1H)

### Synthesis Example 12

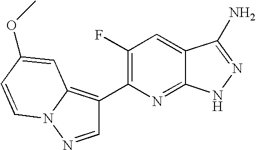

The same procedure as in Synthesis Example 10 was repeated, with the exception that the compound synthesized in Synthesis Example 8 was used as a starting material, to afford the title compound as a bright orange solid.

¹H-NMR (300 MHz, DMSO-d6): 3.06(s, 3H), 5.48(s, 2H), 6.79(dd, J=2.8 Hz, J=7.5 Hz, 1H), 7.95(d, J=11.8 Hz, 1H), 8.17(d, J=2.8 Hz, 1H), 8.46(d, J=4.2 Hz, 1H), 8.69(d, J=7.5 Hz, 1H), 11.93(s, 1H)

### Synthesis Example 13

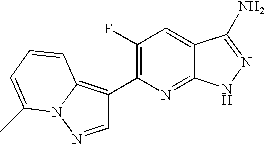

The same procedure as in Synthesis Example 10 was repeated, with the exception that the compound synthesized in Synthesis Example 9 was used as a starting material, to afford the title compound as a bright orange solid.

¹H-NMR (300 MHz, DMSO-d6): 2.76(s, 3H), 5.52(s, 2H), 7.03(d, J=6.9 Hz, 1H), 7.45(dd, J=6.9 Hz, 1H), 8.02(d, J=11.8 Hz, 1H), 8.59(d, J=4.3 Hz, 1H), 8.65(d, J=8.9 Hz, 1H), 11.96(s, 1H)

### Synthesis Example 14

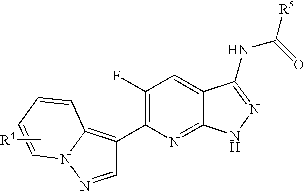

wherein R₄ and R₅ are as defined as in Chemical Formula 1.

To a stirred solution of 5-fluoro-6-pyrazolo[1,5-a]pyridin-3-yl-1H-pyrazolo[3,4-b]pyridin-3-yl amine derivative (1.11 mmol) in pyridine was added acyl chloride (1.67 mmol). The reaction mixture was refluxed overnight, evaporated to concentrate the solvent, and 1N HCl was added thereto. The residue was extracted using ethyl acetate, and the organic phase was dried over Na₂SO₄ and concentrated, followed by purification through recrystallization in a suitable solvent.

Typical examples of the compounds represented by Chemical Formula 2, prepared according to Reaction Scheme 2, are summarized in Table 1, below. In Table 1, M stands for molecular weight, and M+H represents mass spectrum values measured using a mass spectrophotometer (ESI-MS).

TABLE 1

| Prep. | | | |
|---|---|---|---|
| Exampe No. | Molecurlar Structure | [M] | [M + H]+ |
| 1-1 | 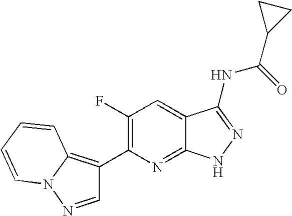 | 336 | 337 |
| 1-3 | 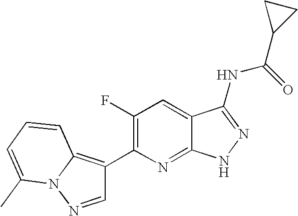 | 350 | 351 |
| 1-5 | 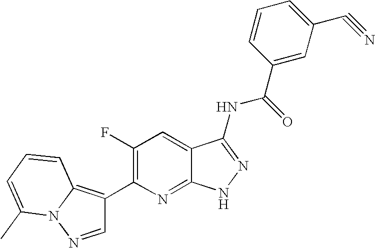 | 411 | 412 |
| 1-7 | 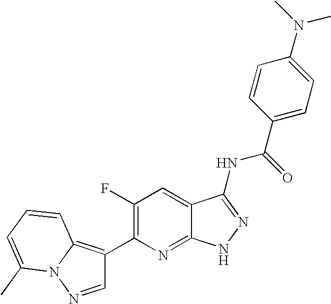 | 429 | 430 |
| 1-9 | 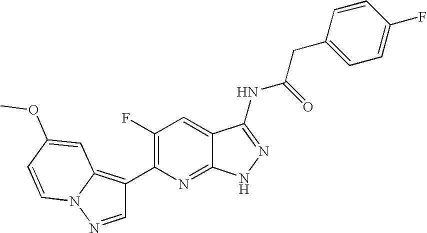 | 434 | 435 |
| 1-11 | 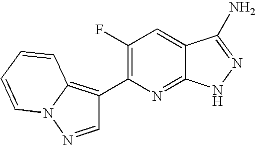 | 268 | 269 |
| 1-13 | 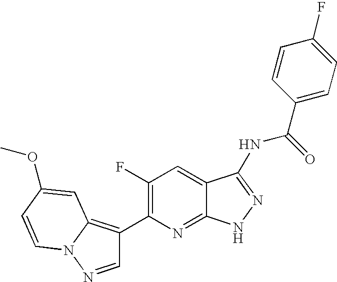 | 420 | 421 |
| 1-15 | 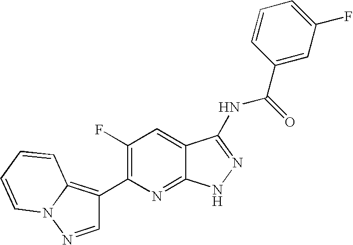 | 390 | 391 |
| 1-17 | 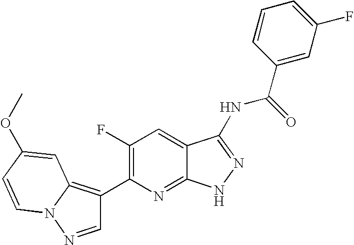 | 420 | 421 |
| 1-19 | 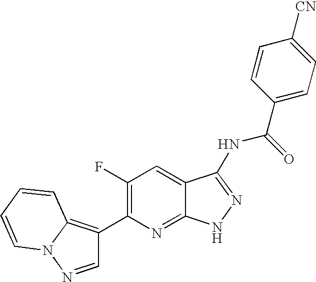 | 397 | 398 |
| 1-21 | 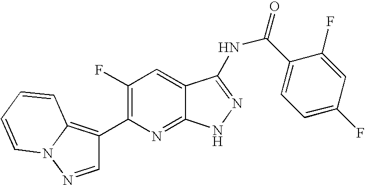 | 408 | 409 |
| 1-23 | 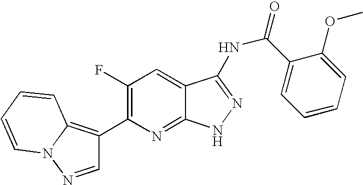 | 402 | 403 |
| 1-25 | 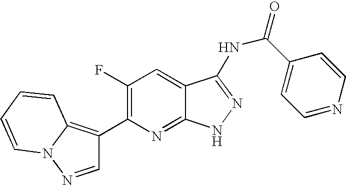 | 373 | 374 |
| 1-27 | 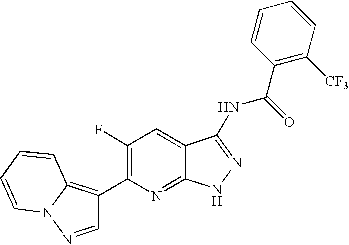 | 440 | 441 |
| 1-29 | 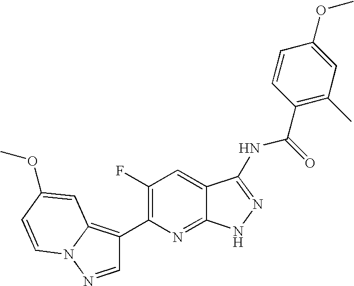 | 446 | 447 |
| Prep. | | | |
| Exampe No. | Molecular Structure | [M] | [M + H]+ |
| 1-2 | 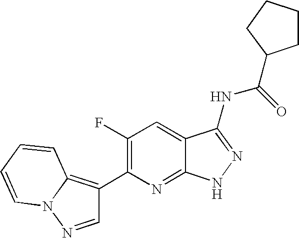 | 364 | 365 |
| 1-4 | 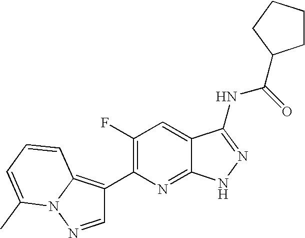 | 378 | 379 |
| 1-6 | 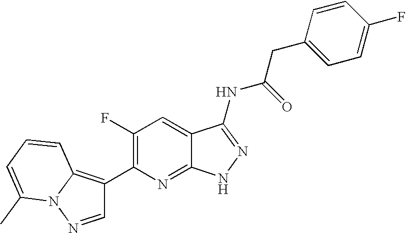 | 418 | 419 |
| 1-8 | 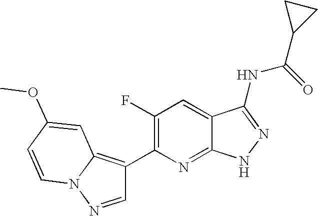 | 366 | 367 |
| 1-10 | 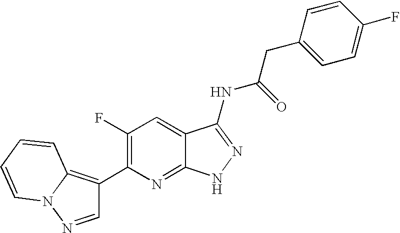 | 404 | 405 |
| 1-12 | 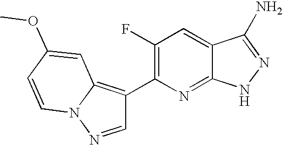 | 298 | 299 |
| 1-14 | 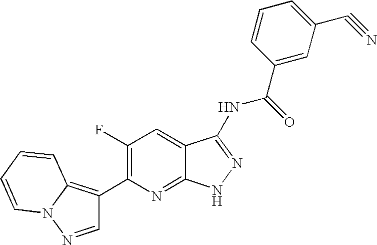 | 397 | 398 |
| 1-16 | 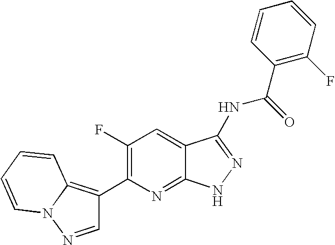 | 390 | 391 |
| 1-18 | 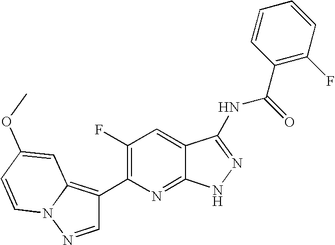 | 420 | 421 |
| 1-20 | 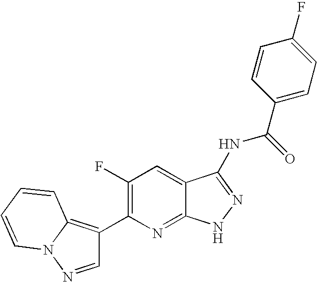 | 390 | 391 |
| 1-22 | 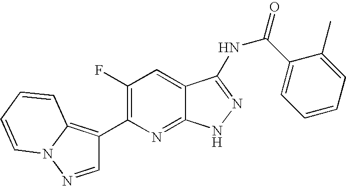 | 386 | 387 |
| 1-24 | 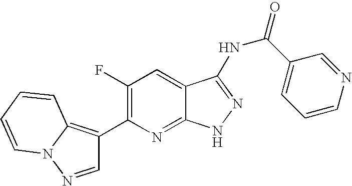 | 373 | 374 |
| 1-26 | 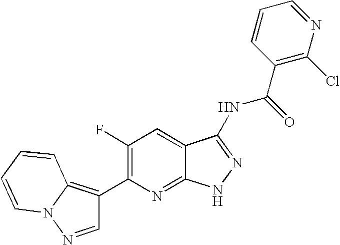 | 407 | 408 |
| 1-28 | 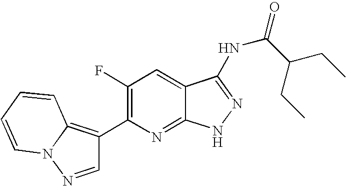 | 366 | 367 |

The preparation examples of the compounds listed in Table 2 is explained below:

### Preparation Example 1-1

### N-(5-fluoro-6-(H-pyrazolo[1,5-a]pyridin-3-yl)-1H-pyrazolo[3,4-b]pyridin-3-yl)cyclopropanecarboxamide

¹H-NMR (300 MHz, DMSO-d6): 0.82-0.89(m, 4H), 1.92-2.01(m, 1H), 7.13(dd, J=6.8 Hz, 1H), 7.55(dd, J=8.9 Hz, 1H), 8.20(d, J=12.5 Hz, 1H), 8.61(d, J=4.2 Hz, 1H), 8.72(d, J=8.9 Hz, 1H), 8.89(d, J=7 Hz, 1H), 11.02(s, 1H), 13.18(s, 1H)

### Preparation Example 1-2

### N-(5-fluoro-6-(H-pyrazolo[1,5-a]pyridin-3-yl)-1H-pyrazolo[3,4-b]pyridin-3-yl)cyclopentanecarboxamide

¹H-NMR (300 MHz, DMSO-d6): 1.55-1.58(m, 2H), 1.62-1.81(m, 4H), 1.83-1.91(m, 2H), 2.93(q, J=7.8 Hz, 1H), 7.15(dd, J=6.8 Hz, 1H), 7.58(dd, J=6.9 Hz, 1H), 8.20(d, J=8.4 Hz, 1H), 8.60(d, J=4.2 Hz, 1H), 8.72(d, J=9 Hz, 1H), 8.88(d, J=6.9 Hz, 1H), 10.66(s, 1H), 13.17(s, 1H)

### Preparation Example 1-3

### N-(5-fluoro-6-(7-methylH-pyrazolo[1,5-a]pyridin-3-yl)-1H-pyrazolo[3,4-b]pyridin-3-yl)cyclopropanecarboxamide

¹H-NMR (300 MHz, DMSO-d6): 0.84-0.89(m, 2H), 1.96-2.01(m, 1H), 2.77(s, 3H), 7.07(d, J=6.9 Hz, 1H), 7.53(dd, J=7 Hz, 1H), 8.19(d, J=12.5 Hz, 1H), 8.63(d, J=4.4 Hz, 1H), 8.69(d, J=9.1 Hz, 1H), 11.01(s, 1H), 13.17(s, 1H)

### Preparation Example 1-4

### N-(5-fluoro-6-(7-methylH-pyrazolo[1,5-a]pyridin-3-yl)-1H-pyrazolo[3,4-b]pyridin-3-yl)cyclopentanecarboxamide

¹H-NMR (300 MHz, DMSO-d6): 1.55-1.59(m, 2H), 1.61-1.79(m, 4H), 1.81-1.91(m, 2H), 2.74(s, 3H), 2.93(q, J=7.6 Hz, 1H), 7.02(d, J=6.9 Hz, 1H), 7.46(dd, J=7.1 Hz, 1H), 8.17(d, J=12.5 Hz, 1H), 8.60(d, J=4.3 Hz, 1H), 8.63(d, J=8.9 Hz, 1H), 10.66(s, 1H), 13.14(s, 1H)

### Preparation Example 1-5

### 3-cyano-N-(5-fluoro-6-(7-methylH-pyrazolo[1,5-a]pyridin-3-yl)-1H-pyrazolo[3,4-b]pyridin-3-yl)benzamide

¹H-NMR (300 MHz, DMSO-d6): 2.78(s, 3H), 7.10 (d, J=6.8 Hz, 1H), 7.54(dd, J=7 Hz, 1H), 7.78(d, J=7.8 Hz, 1H), 8.09(d, J=7.7 Hz, 1H), 8.24(d, J=12.2 Hz, 1H), 8.36(d, J=7.9 Hz, 1H), 8.52(s, 1H), 8.66-8.71(m, 2H), 11.34(s, 1H), 13.42(s, 1H)

### Preparation Example 1-6

### N-(5-fluoro-6-(7-methylH-pyrazolo[1,5-a]pyridin-3-yl)-1H-pyrazolo[3,4-b]pyridin-3-yl)-2-(4-fluorophenyl)acetamide

¹H-NMR (300 MHz, CDCl₃): 2.77(s, 3H), 3.75(s, 2H), 7.07(d, J=6.9 Hz, 1H), 7.17(dd, J=6.8 Hz, 1H), 7.42(dd, J=5.7 Hz, 1H), 7.52(d, J=7 Hz, 1H), 8.16(d, J=12.4 Hz, 1H), 8.65(dd, J=10.3 Hz, 2H), 10.99(s, 1H), 13.29(s, 1H)

### Preparation Example 1-7

### 4-(dimethylamino)-N-(5-fluoro-6-(7-methylH-pyrazolo[1,5-a]pyridin-3-yl)-1H-pyrazolo[3,4-b]pyridin-3-yl)benzamide

¹H-NMR (300 MHz, CDCl₃): 2.77(s, 3H), 3.01(s, 6H), 6.75(d, J=9 Hz, 2H), 7.06(d, J=6.8 Hz, 1H), 7.53(dd, J=7 Hz, 1H), 7.99(d, J=8.8 Hz, 2H), 8.16(d, J=12.3 Hz, 1H), 8.65(d, J=4.2 Hz, 1H), 8.68(d, J=8.9 Hz, 1H), 10.71(s, 1H), 13.25(s, 1H)

### Preparation Example 1-8

### N-(5-fluoro-6-(5-methoxyH-pyrazolo[1,5-a]pyridin-3-yl)-1H-pyrazolo[3,4-b]pyridin-3-yl)cyclopropanecarboxamide

¹H-NMR (300 MHz, CDCl₃): 0.86-0.92(m, 4H), 1.91-1.99(m, 1H), 3.99(s, 3H), 6.82(dd, J=2.7 Hz, J=7.6 Hz, 1H), 8.16-8.22(m, 2H), 8.50(d, J=4.3 Hz, 1H), 8.72(d, J=7.6 Hz, 1H), 11.02(s, 1H), 13.14(s, 1H)

### Preparation Example 1-9

### N-(5-fluoro-6-(5-methoxyH-pyrazolo[1,5-a]pyridin-3-yl)-1H-pyrazolo[3,4-b]pyridin-3-yl)-2-(4-fluorophenypacetamide

¹H-NMR (300 MHz, CDCl₃): 3.71(s, 2H), 3.94(s, 3H), 6.78(dd, J=2.8 Hz, J=7.5 Hz, 1H), 7.13(d, J=8.8 Hz, 2H), 7.38(dd, J=5.7 Hz, 2H), 8.09(d, J=12.6 Hz, 1H), 8.16(d, J=2.8 Hz, 1H), 8.45(d, J=4.3 Hz, 1H), 8.67(d, J=7.5 Hz, 1H), 10.95(s, 1H), 13.16(s, 1H)

### Preparation Example 1-10

### N-(5-fluoro-6-(H-pyrazolo[1,5-a]pyridin-3-yl)-1H-pyrazolo[3,4-b]pyridin-3-yl)-2-(4-fluorophenyl)acetamide

¹H-NMR (300 MHz, CDCl₃): 3.75(s, 2H), 7.11-7.17(m, 3H), 7.42(d, J=5.8 Hz, 1H), 7.45(d, J=5.9 Hz, 1H), 7.57(d, J=6.9 Hz, 1H), 8.16(d, J=8.4 Hz, 1H), 8.59(d, J=4.2 Hz, 1H), 8.71(d, J=9 Hz, 1H), 8.86(d, J=6.9 Hz, 1H), 10.99(s, 1H), 13.23(s, 1H)

### Preparation Example 1-11

### 5-fluoro-6-(H-pyrazolo[1,5-a]pyridin-3-yl)-1H-pyrazolo[3,4-b]pyridin-3-amine

¹H-NMR (300 MHz, DMSO-d6): 5.53(brs, 2H), 7.11(dd, J=6.9 Hz, J=8.9 Hz, 1H), 7.52(dd, J=6.4 Hz, J=6.8 Hz, 1H), 8.02(d, J=11.8 Hz, 1H), 8.55(d, J=4.2 Hz, 1H), 8.68(d, J=9 Hz, 1H), 8.82(d, J=7 Hz, 1H), 11.98(brs, 1H)

### Preparation Example 1-12

### 5-fluoro-6-(5-methoxyH-pyrazolo[1,5-a]pyridin-3-yl)-1H-pyrazolo[3,4-b]pyridin-3-amine

¹H-NMR (300 MHz, DMSO-d6): 3.06(s, 3H), 5.48(s, 2H), 6.79(dd, J=2.8 Hz, J=7.5 Hz, 1H), 7.95(d, J=11.8 Hz, 1H), 8.17(d, J=2.8 Hz, 1H), 8.46(d, J=4.2 Hz, 1H), 8.69(d, J=7.5 Hz, 1H), 11.93(s, 1H)

### Preparation Example 1-13

### 4-fluoro-N-(5-fluoro-6-(5-methoxyH-pyrazolo[1,5-a]pyridin-3-yl)-1H-pyrazolo[3,4-b]pyridin-3-yl)benzamide

¹H-NMR (300 MHz, DMSO-d6): 4.00 (s, 3H), 6.86(dd, J=2.7 Hz, J=7.6 Hz, 1H), 7.39 (t, J=8.7 Hz, 2H), 8.14-8.20(m, 2H), 8.24 (d, J=2.7 Hz, 1H), 8.54 (d, J=4.2 Hz, 1H), 8.76 (d, J=7.5 Hz, 1H), 11.19 (s, 1H), 13.36 (s, 1H)

### Preparation Example 1-14

### 3-cyano-N-(5-fluoro-6-(H-pyrazolo[1,5-a]pyridin-3-yl)-1H-pyrazolo[3,4-b]pyridin-3-yl)benzamide

¹H-NMR (300 MHz, CDCl₃): 7.14(dd, J=6.8 Hz, 1H), 7.57(dd, J=6.9 Hz, 1H), 7.78 (dd, J=7.9 Hz, 1H), 8.09(d, J=7.7 Hz, 1H), 8.21(d, J=12.1 Hz, 1H), 8.36(d, J=8 Hz, 1H),8.52(s, 1H), 8.63(d, J=4.2 Hz, 1H), 8.75(d, J=8.9 Hz, 1H), 8.87(d, J=6.8 Hz, 1H), 11.35 (s, 1H), 13.43(s, 1H)

### Preparation Example 1-15

### 3-fluoro-N-(5-fluoro-6-(H-pyrazolo[1,5-a]pyridin-3-yl)-1H-pyrazolo[3,4-b]pyridin-3-yl)benzamide

¹H-NMR (300 MHz, DMSO-d6) δ 7.16(dd, J=7.2 Hz, 1H), 7.35-7.4(m, 1H), 7.48(ddd, J=8.7, 2.1 Hz, 1H), 7.57-7.65(m, 2H), 7.78(m, 1H), 8.2(d, J=12.1 Hz, 1H), 8.63(d, J=4.2 Hz, 1H), 8.77(d, J=8.9 Hz, 1H), 8.89(d, J=6.8 Hz, 1H), 11.23(s, 1H), 13.44(bs, 1H)

### Preparation Example 1-16

### 2-fluoro-N-(5-fluoro-6-(H-pyrazolo[1,5-a]pyridin-3-yl)-1H-pyrazolo[3,4-b]pyridin-3-yl)benzamide

¹H-NMR (300 MHz, DMSO-d6) δ 7.17(dd, J=6.8 Hz, 1H), 7.36(m, 2H), 7.6 (m, 2H), 7.78(ddd, J=7.4, 1 Hz, 1H), 8.26(d, J=12 Hz, 1H), 8.64(d, J=4.23 Hz, 1H), 8.76(d, J=8.8 Hz, 1H), 8.89(d, J=6.9 Hz, 1H)

### Preparation Example 1-17

### 3-fluoro-N-(5-fluoro-6-(5-methoxyH-pyrazolo[1,5-a]pyridin-3-yl)-1H-pyrazolo[3,4-b]pyridin-3-yl)benzamide

¹H-NMR (300 MHz, DMSO-d6): 4.03 (s, 3H), 6.91(dd, J=2.6 Hz, J=7.6 Hz, 1H), 7.39 (t, J=8.7 Hz, 2H), 7.92-8.21 (m, 3H), 8.22 (d, J=4.2 Hz, 1H), 8.54 (d, J=4.2 Hz, 1H), 8.76 (d, J=7.5 Hz, 1H), 11.03 (s, 1H), 13.12 (s, 1H)

### Preparation Example 1-18

### 2-fluoro-N-(5-fluoro-6-(5-methoxyH-pyrazolo[1,5-a]pyridin-3-yl)-1H-pyrazolo[3,4-b]pyridin-3-yl)benzamide

¹H-NMR (300 MHz, DMSO-d6): 3.98 (s, 3H), 6.85(dd, J=2.4 Hz, J=7.4 Hz, 1H), 7.32-7.41 (m, 3H), 7.92-8.21 (m, 2H), 8.22 (d, J=4.2 Hz, 1H), 8.54 (d, J=4.2 Hz, 1H), 8.76 (d, J=7.5 Hz, 1H), 11.12(s, 1H), 13.43 (s, 1H)

### Preparation Example 1-19

### 4-cyano-N-(5-fluoro-6-(H-pyrazolo[1,5-a]pyridin-3-yl)-1H-pyrazolo[3,4-b]pyridin-3-yl)benzamide

¹H-NMR (300 MHz, DMSO-d6): 7.16 (d, J=6.9 Hz, 1H), 7.59 (t, J=7.5 Hz, 1H), 8.04 (d, J=8.1 Hz, 2H), 8.19-8.25 (m, 3H), 8.62 (d, J=4.2 Hz, 1H), 8.76 (d, J=9.0 Hz, 1H), 8.88 (d, J=6.9 Hz, 1H), 11.43 (s, 1H), 13.48 (s, 1H)

### Preparation Example 1-20

### 4-fluoro-N-(5-fluoro-6-(H-pyrazolo[1,5-a]pyridin-3-yl)-1H-pyrazolo[3,4-b]pyridin-3-yl)benzamide

¹H-NMR (300 MHz, DMSO-d6): 7.16 (d, J=6.9 Hz, 1H), 7.36-7.44 (m, 2H), 7.60 (t, J=6.6 Hz, 1H), 8.15-8.22 (m, 2H), 8.59 (d, J=4.2 Hz, 1H), 8.63 (d, J=4.2 Hz, 1H), 8.76 (d, J=9.0 Hz, 1H), 8.89 (d, J=6.9 Hz, 1H), 11.17 (s, 1H), 13.40 (s, 1H)

### Preparation Example 1-21

### 2,4-difluoro-N-(5-fluoro-6-(H-pyrazolo[1,5-a]pyridin-3-yl)-1H-pyrazolo[3,4-b]pyridin-3-yl)benzamide

¹H-NMR (300 MHz, DMSO-d6): 7.17(t, J=6.9 Hz, 1H), 7.27 (t, J=8.7 Hz, 1H), 7.42-7.48 (m, 1H), 7.61 (t, J=6.9 Hz, 1H), 7.83-7.92 (m, 1H), 8.27 (d, J=12.3 Hz, 1H), 8.64 (d, J=4.2 Hz, 1H), 8.77 (d, J=8.7 Hz, 1H), 8.90(d, J=6.9 Hz, 1H), 11.17 (s, 1H), 13.40 (s, 1H)

### Preparation Example 1-22

### N-(5-fluoro-6-(H-pyrazolo[1,5-a]pyridin-3-yl)-1H-pyrazolo]3,4-b]pyridin-3-yl)-2-methylbenzamide

¹H-NMR (300 MHz, DMSO-d6): 2.47 (s, 3H), 7.16(t, J=6.9 Hz, 1H), 7,28-7.34 (m, 1H), 7.40-7.45 (m, 1H), 7.59 (t, J=6.9 Hz, 1H), 7.84 (t, J=7.8 Hz, 1H), 8.24 (d, J=12.3 Hz, 1H), 8.59 (d, J=4.2 Hz, 1H), 8.63 (d, J=4.2 Hz, 1H), 8.77 (d, J=9.0 Hz, 1H), 8.89(d, J=6.9 Hz, 1H), 11.02 (s, 1H), 13.34 (s, 1H)

### Preparation Example 1-23

### N-(5-fluoro-6-(H-pyrazolo[1,5-a]pyridin-3-yl)-1H-pyrazolo[3,4-b]pyridin-3-yl)-2-methoxybenzamide

¹H-NMR (300 MHz, DMSO-d6): 3.98 (s, 3H), 7.10-7.19 (m, 2H), 7,24 (d, J=8.4 Hz, 1H), 7.58 (q, J=6.6 Hz, 2H), 7.88 (d, J=7.2 Hz, 1H), 8.33 (d, J=12.6 Hz, 1H), 8.65 (d, J=4.2 Hz, 1H), 8.77 (d, J=9.0 Hz, 1H), 8.89(d, J=6.9 Hz, 1H), 10.65 (s, 1H), 13.37 (s, 1H)

### Preparation Example 1-24

### N-(5-fluoro-6-(H-pyrazolo[1,5-a]pyridin-3-yl)-1H-pyrazolo[3,4-b]pyridin-3-yl)nicotinamide

¹H-NMR (300 MHz, DMSO-d6): 7.17 (t, J=6.9 Hz, 1H), 7.57-7.62 (m, 2H), 8.24 (d, J=12.3 Hz, 1H), 8.42 (d, J=7.8 Hz, 1H), 8.64 (d, J=4.2 Hz, 1H), 8.75-8.80 (m, 2H), 8.89 (d, J=6.9 Hz, 1H), 9.23 (s,1H), 11.37 (s, 1H), 13.43 (s, 1H)

### Preparation Example 1-25

### N-(5-fluoro-6-(H-pyrazolo[1,5-a]pyridin-3-yl)-1H-pyrazolo[3,4-b]pyridin-3-yl)isonicotinamide

¹H-NMR (300 MHz, DMSO-d6): 7.17 (t, J=6.9 Hz, 1H), 7.60 (t, J=7.5 Hz, 1H), 8.00 (d, J=6.0 Hz, 2H), 8.24 (d, J=12.3 Hz, 1H), 8.64 (d, J=4.2 Hz, 1H), 8.77 (d, J=9.0 Hz, 2H), 8.82 (d, J=5.7 Hz, 2H), 8.89 (d, J=6.9 Hz, 1H), 9.23 (s,1H), 11.45 (s, 1H), 13.47 (s, 1H)

### Preparation Example 1-26

### 2-chloro-N-(5-fluoro-6-(H-pyrazolo[1,5-a]pyridin-3-yl)-1H-pyrazolo[3,4-b]pyridin-3-yl)nicotinamide

¹H-NMR (300 MHz, DMSO-d6): 7.17 (t, J=6.9 Hz, 1H), 7.49-7.53 (m, 1H), 7.56-7.62 (m, 1H), 7.94 (t, J=7.5 Hz, 1H), 8.17 (d, J=7.2 Hz, 1H), 8.28 (d, J=12.3 Hz, 1H), 8.55 (d, J=4.5 Hz, 1H), 8.76 (d, J=8.4 Hz, 1H), 8.88 (d, J=6.9 Hz, 1H), 11.46 (s, 1H), 13.44 (s, 1H)

### Preparation Example 1-27

### N-(5-fluoro-6-(H-pyrazolo[1,5-a]pyridin-3-yl)-1H-pyrazolo[3,4-b]pyridin-3-yl)-2-(trifluoromethypbenzamide

¹H-NMR (300 MHz, DMSO-d6): 7.15-7.19 (m, 1H), 7.36-7.40 (m, 1H), 7.57-7.62 (m, 1H), 7.70-7.79 (m, 1H), 7.88 (d, J=7.5 Hz, 1H), 8.17 (d, J=12.3 Hz, 1H), 8.57 (d, J=4.2 Hz, 1H), 8.64 (d, J=4.5 Hz, 1H), 8.76 (d, J=9.0 Hz, 1H), 8.89 (d, J=6.9 Hz, 1H), 11.36 (s, 1H), 13.36 (s, 1H)

### Preparation Example 1-28

### 2-ethyl-N-(5-fluoro-6-(H-pyrazolo[1,5-a]pyridin-3-yl)-1H-pyrazolo[3,4-b]pyridin-3-yl)butanamide

¹H-NMR (300 MHz, DMSO-d6): 0.90 (t, J=7.2 Hz, 6H), 1.45-1.67 (m, 4H), 2.42 (m, 1H), 7.16 (t, J=6.9 Hz, 1H), 7.58 (t, J=6.9 Hz, 1H), 8.18 (d, J=12.3 Hz, 1H), 8.61 (d, J=4.2 Hz, 1H), 8.74 (d, J=9.0 Hz, 1H), 8.88 (d, J=6.9 Hz, 1H), 10.70 (s, 1H), 13.21 (s, 1H)

### Preparation Example 1-29

### N-(5-fluoro-6-(5-methoxyH-pyrazolo[1,5-a]pyridin-3-yl)-1H-pyrazolo[3,4-b]pyridin-3-yl)-4-methoxy-2-methylbenzamide

¹H-NMR (300 MHz, DMSO-d6): 2.47(s, 3H), 3.81(s, 3H), 4.00(s, 3H), 7.38(m, 1H), 7.6(d, J=8.2 Hz, 1H), 8.18(d, J=12.53 Hz, 1H), 8.24(d, J=2.67 Hz, 1H), 8.53(d, J=4.26 Hz, 1H), 8.57(m, 1H) 8.73(s, 1H), 8.76(s, 1H), 10.85(s, 1H), 13,25(s, 1H)

Representative examples of the compounds represented by Chemical Formula 2, prepared according to Reaction Scheme 2, are summarized in Table 2, below. In Table 2, M stands for molecular weight, and M+H represents mass spectrum values measured using a mass spectrophotometer (ESI-MS).

TABLE 2

| Prep. | | | Molecular |
|---|---|---|---|
| Example No. | Structure | [M] | Formula |
| 1-30 | 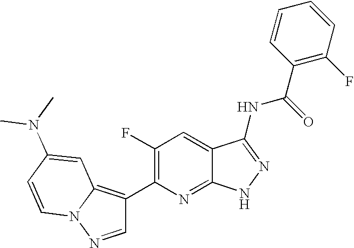 | 433 | C₂₂H₁₇F₂N₇O |
| 1-31 | 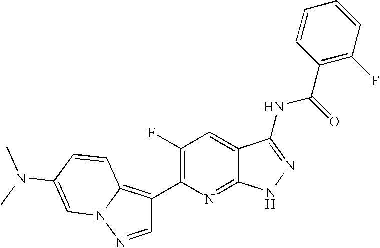 | 433 | C₂₂H₁₇F₂N₇O |
| 1-32 | 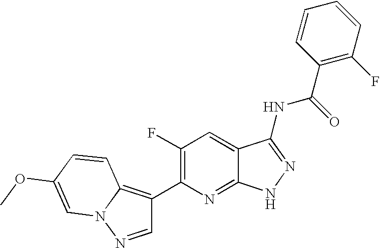 | 420 | C₂₁H₁₄F₂N₆O₂ |
| 1-33 | 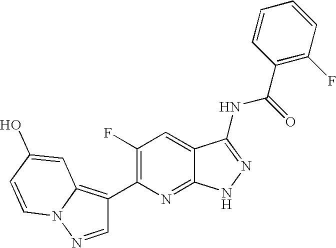 | 406 | C₂₀H₁₂F₂N6O₂ |
| 1-34 | 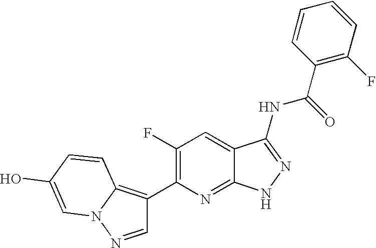 | 406 | C₂₀H₁₂F₂N₆O₂ |
| 1-35 | 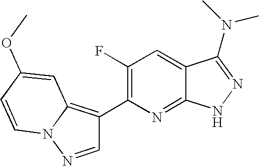 | 326 | C₁₆H₁₅FN₆O |
| 1-36 | 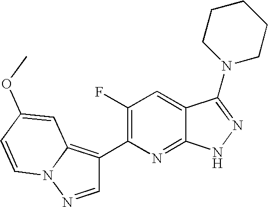 | 366 | C₁₉H₁₉FN₆O |
| 1-37 | 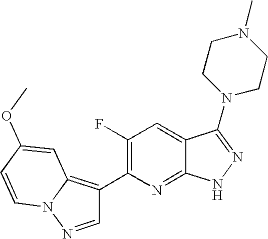 | 381 | C₁₉H₂₀FN₇O |
| 1-38 | 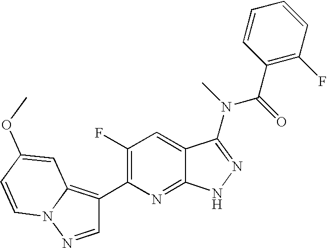 | 434 | C₂₂H₁₆F₂N₆O₂ |
| 1-39 | 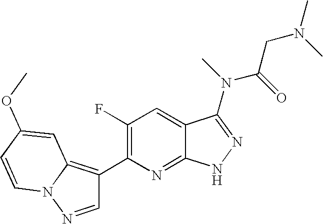 | 397 | C₁₉H₂0FN₇O₂ |
| 1-40 | 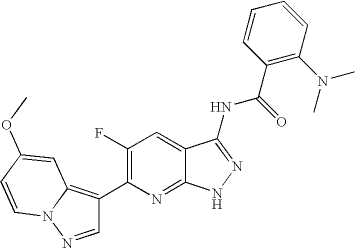 | 445 | C₂₃H₂₀FN₇O₂ |
| 1-41 | 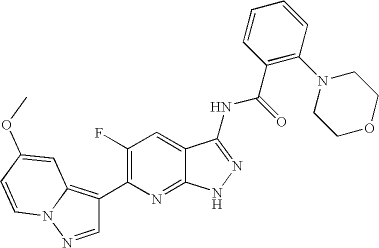 | 487 | C₂₅H₂₂FN₇O₃ |
| 1-42 | 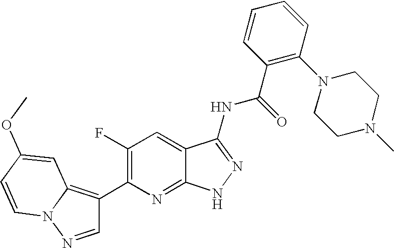 | 500 | C₂₆H₂₅FN₈O₂ |
| 1-43 | 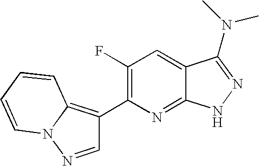 | 296 | C₁₅H₁₃FN₆ |
| 1-44 | 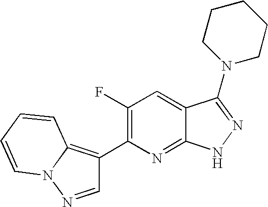 | 336 | C₁₈H₁₇FN₆ |
| 1-45 | 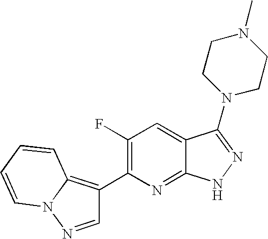 | 351 | C₁₈H₁₈FN₇ |
| 1-46 | 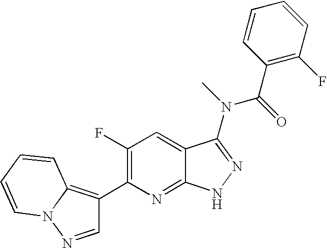 | 404 | C₂₁H₁₄F₂N₆O |
| 1-47 | 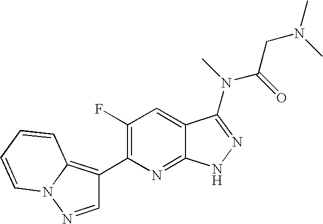 | 367 | C₁₈H₁₈FN₇O |
| 1-48 | 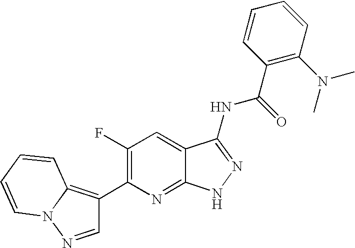 | 415 | C₂₂H₁₈FN₇O |
| 1-49 | 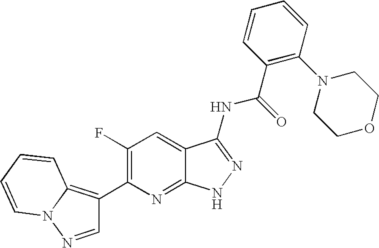 | 457 | C₂₄H₂₀FN₇O₂ |
| 1-50 | 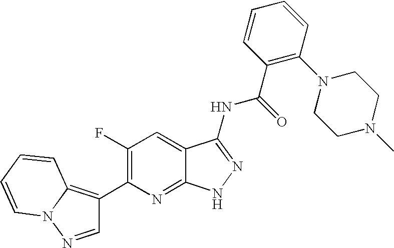 | 470 | C₂₅H₂₃FN₈O |
| 1-51 | 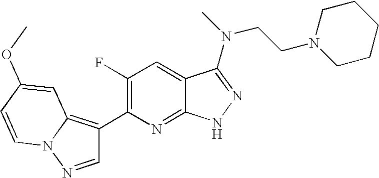 | 423 | C₂₂H₂₆FN₇O |
| 1-52 | 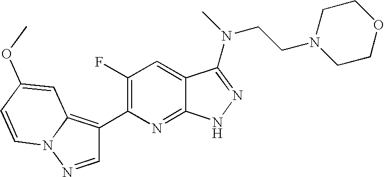 | 425 | C₂₁H₂₄FN₇O₂ |
| 1-53 | 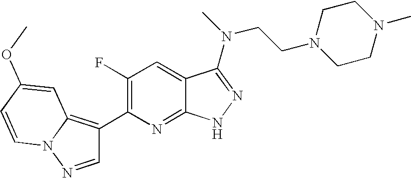 | 438 | C₂₂H₂₇FN₈O |
| 1-54 | 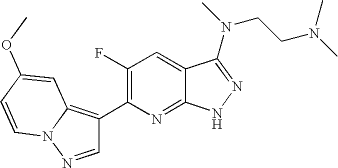 | 383 | C₁₉H₂₂FN₇O |
| 1-55 | 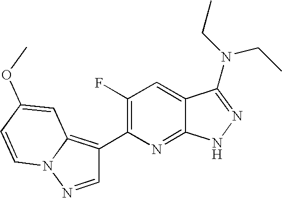 | 354 | C₁₈H₁₉FN₆O |
| 1-56 | 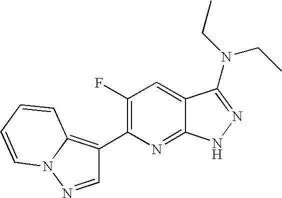 | 324 | C₁₇H₁₇FN₆ |
| 1-57 | 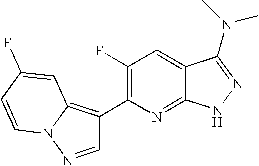 | 314 | C₁₅H₁₂F₂N₆ |
| 1-58 | 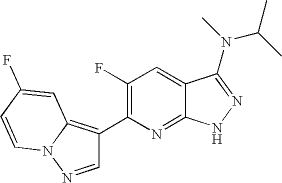 | 342 | C₁₇H₁₆F₂N₆ |
| 1-59 | 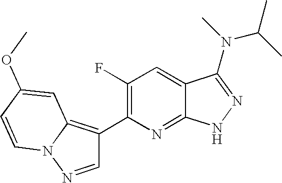 | 354 | C₁₈H₁₉FN₆O |
| 1-70 | 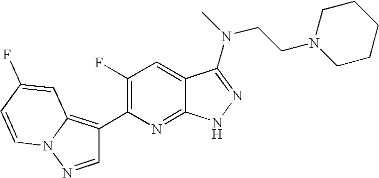 | 411 | C₂₁H₂₃F₂N₇ |
| 1-71 | 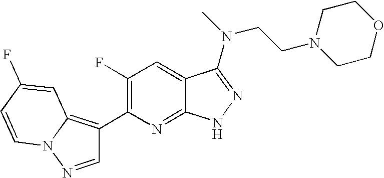 | 413 | C₂₀H₂₁F₂N₇O |
| 1-72 | 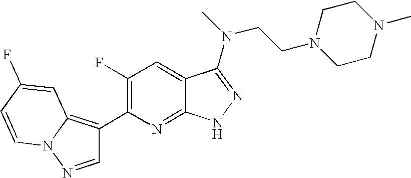 | 426 | C₂₁H₂₄F₂N₈ |
| 1-73 | 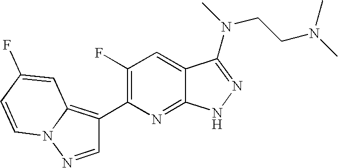 | 371 | C₁₈H₁₉F₂N₇ |
| 1-74 | 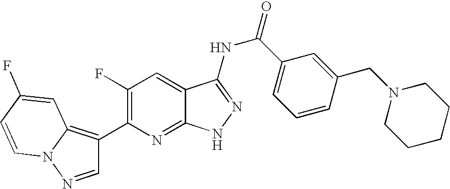 | 487 | C₂₆H₂₃F₂N₇O |
| 1-75 | 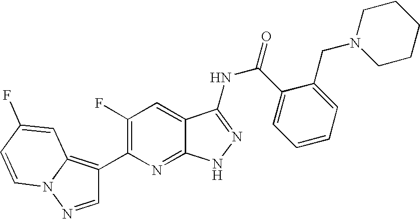 | 487 | C₂₆H₂₃F₂N₇O |
| 1-76 | 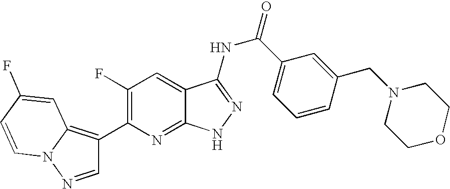 | 489 | C₂₅H₂₁F₂N₇O₂ |
| 1-77 | 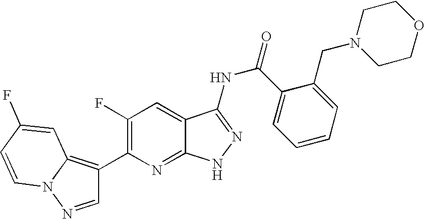 | 489 | C₂₅H₂₁F₂N₇O |
| 1-78 | 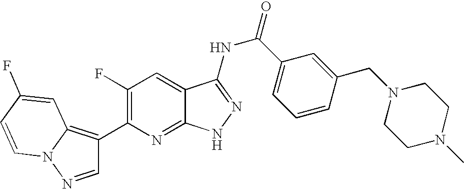 | 502 | C₂₆H₂₄F₂N₈O |
| 1-79 | 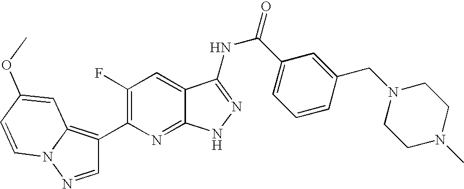 | 514 | C₂₂H₂₇FN₈O₂ |

The synthesis examples according to reaction scheme 2 are described below.

### Synthesis Example 15

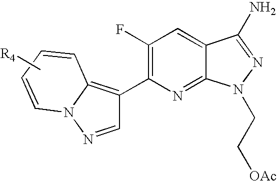

wherein R₄ is as defined as in Chemical Formula 1.

To a solution of the derivative obtained in Synthesis Example 10, serving as a starting material, in DMF was added Cs₂CO₃ (0.035 mol), and the mixture was stirred at room temperature for 30 min and mixed with 2-bromoethyl acetate (0.014 mol). After being heated overnight at 50° C., the reaction mixture was extracted with ethyl acetate and washed with water. The organic phase was dried over Na₂SO₄, and concentrated under vacuum. Column chromatography using ethylacetate/hexane afforded the desired product. Representative examples of the compounds obtained are given in Table 3, below.

### Synthesis Example 16

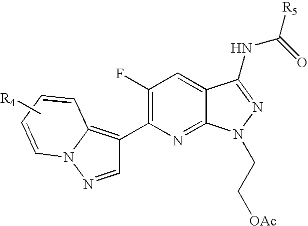

wherein R₄ and R₅ are as defined as in Chemical Formula 1.

To a solution of the derivative obtained in Synthesis Example 15 serving as a starting material in dioxane, were added DIEA (5.249 mmol) and acyl chloride (2.62 mmol). The reaction mixture was stirred at room temperature for 4 hours and mixed with 1N HCl. The precipitate was filtered and washed with Et₂O. The filtrate was dried under vacuum to afford the desired product as a yellow solid. Examples of the compounds obtained are given in Table 3, below.

### Synthesis Example 17

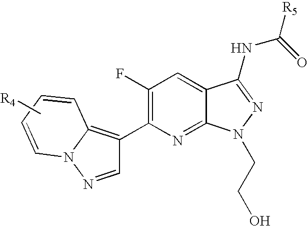

wherein R₄ and R₅ are as defined as in Chemical Formula 1.

To a solution of the derivative obtained in Synthesis Example 16 serving as a starting material in MeOH, was added K₂CO₃ (1.96 mmol). This reaction mixture was stirred at room temperature for 1 hour and the solvent was removed under vacuum. The residue thus obtained was extracted with MC. The organic phase was dried over Na₂SO₄ and concentrated under vacuum to afford the desired product as a solid. Typical examples of the compounds thus obtained are given in Table 3, below.

Typical examples of the compounds represented by Chemical Formula 2, prepared according to Reaction Scheme 2, are described in Table 3, below. In Table 3, M stands for molecular weight, and M+H represents mass spectrum values measured using a mass spectrophotometer (ESI-MS).

TABLE 3

| Prep. | | | |
|---|---|---|---|
| Example No. | Molecular Structure | [M] | [M + H]+ |
| 1-80 | 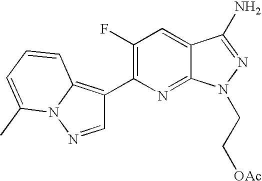 | 368 | 369 |
| 1-81 | 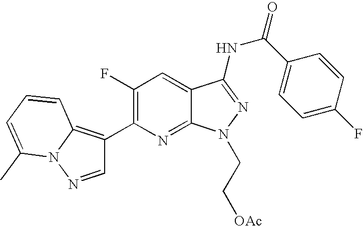 | 490 | 491 |
| 1-82 | 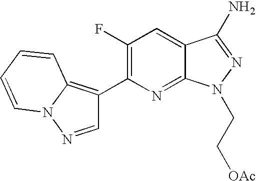 | 354 | 355 |
| 1-83 | 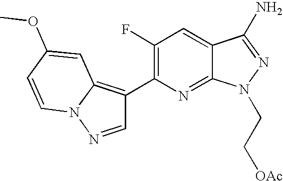 | 384 | 385 |
| 1-84 | 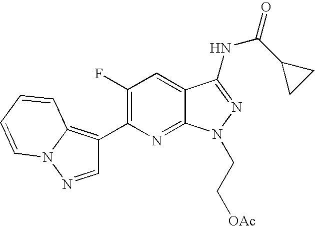 | 422 | 423 |
| 1-85 | 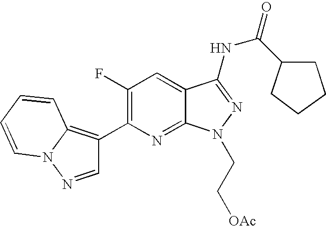 | 450 | 451 |
| 1-86 | 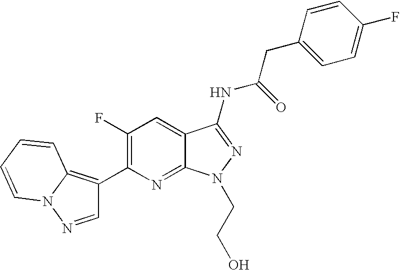 | 448 | 449 |
| 1-87 | 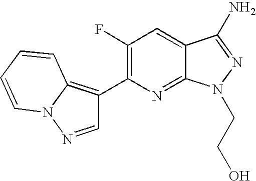 | 312 | 313 |
| 1-88 | 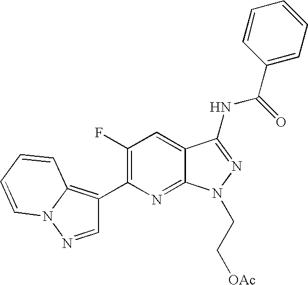 | 458 | 459 |
| 1-89 | 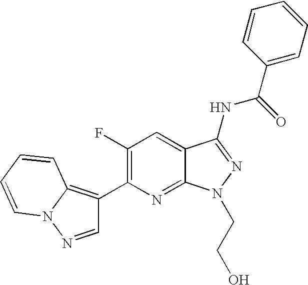 | 416 | 417 |
| 1-90 | 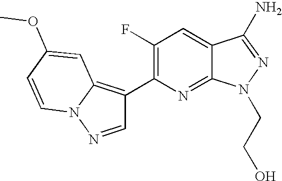 | 342 | 343 |
| 1-91 | 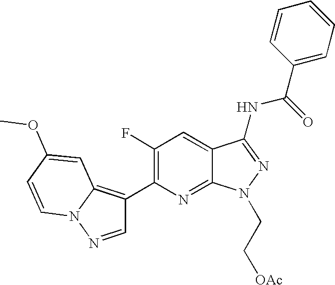 | 488 | 489 |
| 1-92 | 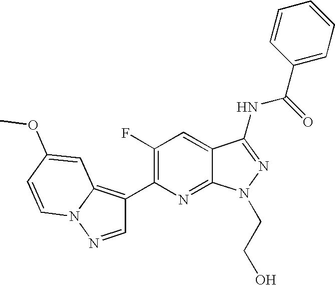 | 446 | 447 |
| 1-93 | 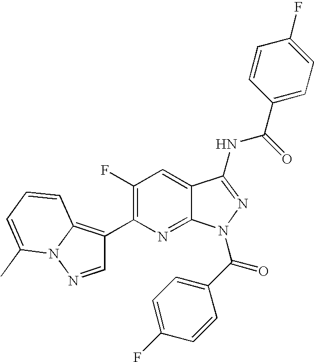 | 526 | 527 |
| 1-94 | 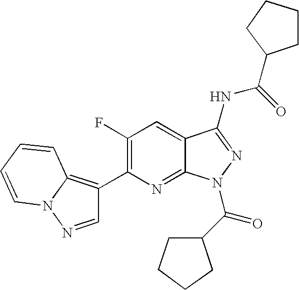 | 460 | 461 |
| 1-95 | 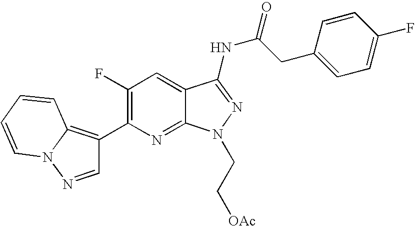 | 490 | 491 |
| 1-96 | 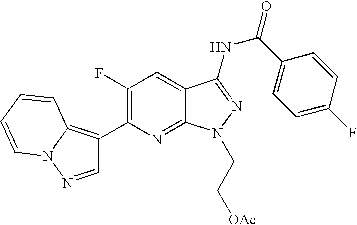 | 476 | 477 |
| 1-97 | 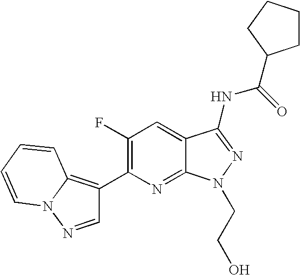 | 408 | 409 |
| 1-98 | 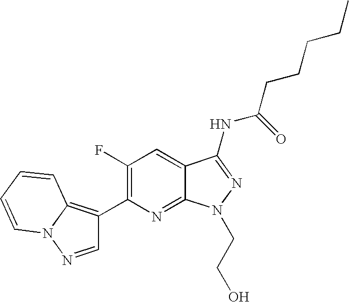 | 410 | 411 |
| 1-99 | 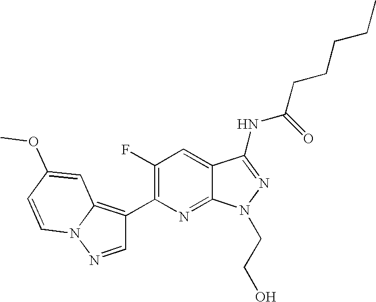 | 440 | 441 |
| 1-100 | 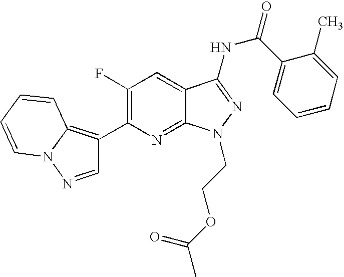 | 472 | 473 |
| 1-101 | 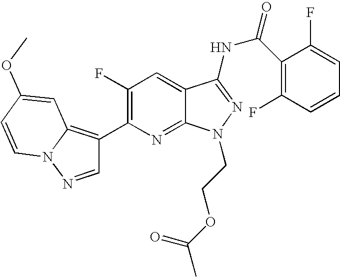 | 524 | 525 |
| 1-102 | 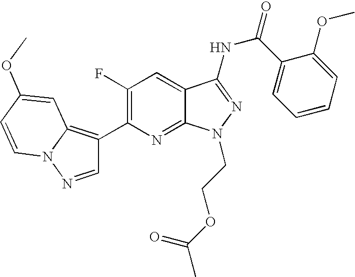 | 518 | 519 |
| 1-103 | 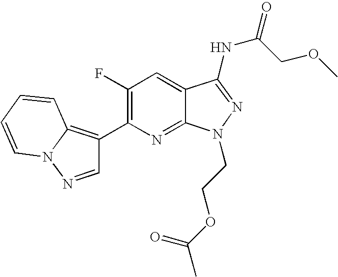 | 426 | 427 |
| 1-104 | 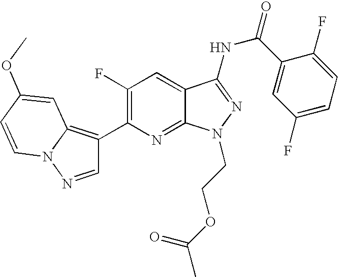 | 524 | 525 |
| 1-105 | 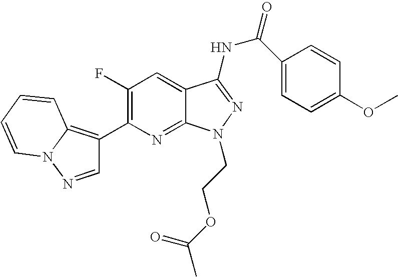 | 488 | 489 |
| 1-106 | 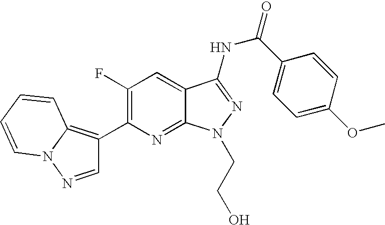 | 446 | 447 |
| 1-107 | 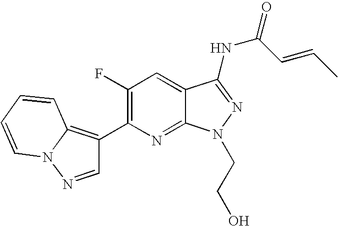 | 380 | 381 |
| 1-108 | 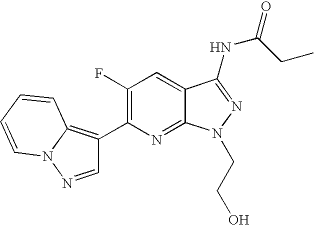 | 368 | 369 |
| 1-109 | 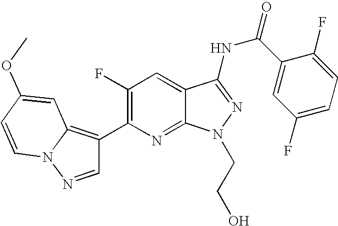 | 482 | 483 |
| 1-110 | 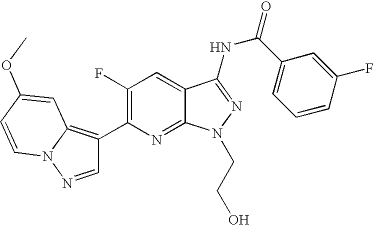 | 464 | 465 |
| 1-111 | 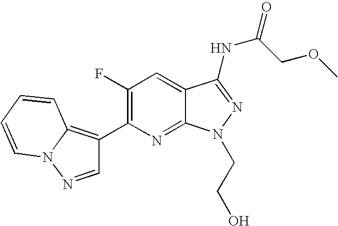 | 384 | 385 |
| 1-112 | 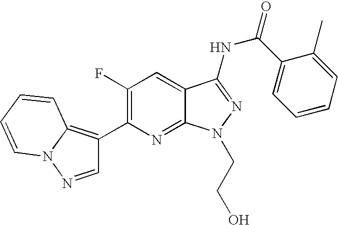 | 430 | 431 |
| 1-113 | 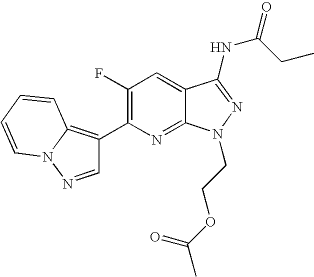 | 410 | 411 |
| 1-114 | 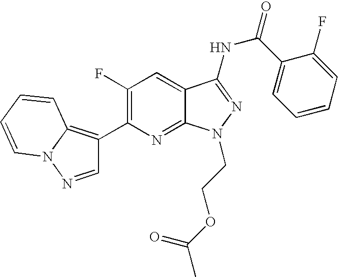 | 476 | 477 |
| 1-115 | 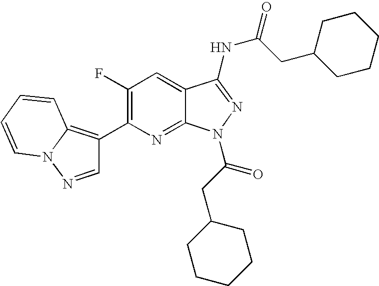 | 516 | 517 |

The preparation examples of the above compounds are described below in detail:
Prep. Example 1-80

### 2-(3-amino-5-fluoro-6-(7-methylH-pyrazolo[1,5-a]pyridin-3-yl)-1H-pyrazolo[3,4-b]pyridin-1-yl)ethyl acetate

¹H-NMR (300 MHz, CDCl₃): 1.83(s, 3H), 2.84(s, 3H), 4.04(s, 2H), 4.51(t, J=5.4 Hz, 2H), 4.63(t, J=5.2 Hz, 2H), 6.83(d, J=6.8 Hz, 1H), 7.37(dd, J=7 Hz, 1H), 7.56(d, J=11.1 Hz, 1H), 8.70(d, J=4.2 Hz, 1H), 8.79(d, J=8.9 Hz, 1H)

### Prep. Example 1-81

### 2-(5-fluoro-3-(4-fluorobenzamido)-6-(7-methylH-pyrazolo[1,5-a]pyridin-3-yl)-1H-pyrazolo[3,4-b]pyridin-1-yl)ethyl acetate

¹H-NMR (300 MHz, CDCl₃): 1.85(s, 3H), 2.85(s, 3H), 4.57(t, J=5.3 Hz, 1H), 4.75(t, J=5.4 Hz, 1H), 6.87(d, J=6.9 Hz, 1H), 7.19(d, J=8.5 Hz, 1H), 7.39(dd, J=7 Hz, 1H), 7.99(dd, J=5.2 Hz, J=8.8 Hz, 1H), 8.38(d, J=12.1 Hz, 1H), 8.52(s, 1H), 8.75(dd, J=4.2 Hz, 1H)

### Prep. Example 1-82

### 2-(3-amino-5-fluoro-6-(H-pyrazolo[1,5-a]pyridin-3-yl)-1H-pyrazolo[3,4-b]pyridin-1-yl)ethyl acetate

¹H-NMR (300 MHz, CDCl₃): 1.83(s, 3H), 4.06(s, 2H), 4.53(t, J=5.2 Hz, 1H), 4.60(t, J=5.1 Hz, 1H), 6.96(dd, J=6.8 Hz, 1H), 7.43(dd, J=7.8 Hz, 1H), 7.56(d, J=11.1 Hz, 1H), 8.55(d, J=7 Hz, 1H), 8.65(d, J=4.2 Hz, 1H), 8.79(dd, J=8.9 Hz, 1H)

### Prep. Example 1-83

### 2-(3-amino-5-fluoro-6-(5-methoxyH-pyrazolo[1,5-a]pyridin-3-yl)-1H-pyrazolo[3,4-b]pyridin-1-yl)ethyl acetate

¹H-NMR (300 MHz, CDCl₃): 1.79(s, 3H), 4.02(s, 3H), 4.05(s, 2H), 4.52(t, J=5.1 Hz, 1H), 4.59(t, J=5.0 Hz, 1H), 6.62(dd, J=2.8 Hz, J=7.5 Hz, 1H), 7.53(d, J=11.2 Hz, 1H), 8.16(d, J=2.8 Hz, 1H), 8.36(d, J=7.6 Hz, 1H), 8.57(d, J=4.3 Hz, 1H)

### Prep. Example 1-84

### 2-(3-(cyclopropanecarboxamido)-5-fluoro-6-(H-pyrazolo[1,5-a]pyridin-3-yl)-1H-pyrazolo[3,4-b]pyridin-1-yl)ethyl acetate

¹H-NMR (300 MHz, DMSO-d6): 0.85-0.93(m, 4H), 1.76(s, 3H), 1.96-1.98(m, 1H), 4.49(t, J=4.9 Hz, 1H), 4.70(t, J=4.9 Hz, 1H), 7.17(dd, J=6.8 Hz, 1H), 7.59(t, J=7.2 Hz, 1H), 8.22(d, J=12.5 Hz, 1H), 8.62(d, J=4.0 Hz, 1H), 8.77(d, J=8.9 Hz, 1H), 8.87(d, J=6.9 Hz, 1H),

### Prep. Example 1-85

### 2-(3-(cyclopentanecarboxamido)-5-fluoro-6-(H-pyrazolo[1,5-a]pyridin-3-yl)-1H-pyrazolo[3,4-b]pyridin-1-yl)ethyl acetate

¹H-NMR (300 MHz, DMSO-d6): 1.56-1.59(m, 2H), 1.64-1.81(m, 7H), 1.89-1.91(m, 2H), 2.91-2.96(m, 1H), 4.49(t, J=5 Hz, 2H), 4.70(t, J=5 Hz, 2H), 7.17(dd, J=6.8 Hz, 1H), 7.59(dd, J=7.6 Hz, 1H), 8.24(d, J=12.4 Hz, 1H), 8.63(d, J=2.1 Hz, 1H), 8.78(dd, J=8.9 Hz, 1H), 8.87(dd, J=6.9 Hz, 1H), 10.8(s, 1H)

### Prep. Example 1-86

### N-(5-fluoro-1-(2-hydroxyethyl)-6-(H-pyrazolo[1,5-a]pyridin-3-yl)-1H-pyrazolo[3,4-b]pyridin-3-yl)-2-(4-fluorophenyl)acetamide

¹H-NMR (300 MHz, DMSO-d6): 3.75(s, 2H), 3.91(s, 2H), 4.50(t, J=5.5 Hz, 2H), 7.14-7.20(m, 3H), 7.41(d, J=5.6 Hz, 1H), 7.42(d, J=5.8 Hz, 1H), 7.60(dd, J=8.6 Hz, 1H), 8.19(d, J=12.4 Hz, 1H), 8.63(d, J=4.3 Hz, 1H), 8.78(d, J=8.9 Hz, 1H), 8.89(d, J=6.9 Hz, 1H), 11.10(s, 1H)

### Prep. Example 1-87

### 2-(3-amino-5-fluoro-6-(H-pyrazolo[1,5-a]pyridin-3-yl)-1H-pyrazolo[3,4-b]pyridin-1-yl)ethanol

¹H-NMR (300 MHz, DMSO-d6): 3.81(t, J=6.0 Hz, 2H), 4.28(t, J=6.1 Hz, 2H), 4.81(s, 1H), 5.61(s, 2H), 7.12(dd, J=6.8 Hz, 1H), 7.55(dd, J=6.8 Hz, 1H), 8.02(d, J=11.7 Hz, 1H), 8.56(d, J=4.3 Hz, 1H), 8.74(d, J=8.9 Hz, 1H), 8.83(d, J=6.9 Hz, 1H)

### Prep. Example 1-88

### 2-(3-benzamido-5-fluoro-6-(H-pyrazolo[1,5-a]pyridin-3-yl)-1H-pyrazolo[3,4-b]pyridin-1-yl)ethyl acetate

¹H-NMR (300 MHz, CDCl₃): 1.86(s, 3H), 4.59(t, J=5.3 Hz, 2H), 4.76(t, J=5.4 Hz, 2H), 6.99(dd, J=6.9 Hz, 1H), 7.44-7.62(m, 4H), 7.99(d, J=6.9 Hz, 2H), 8.45(d, J=12 Hz, 1H), 8.57-8.60(m, 2H), 8.72(d, J=4.1 Hz, 1H), 8.81(d, J=8.9 Hz, 1H)

### Prep. Example 1-89

### N-(5-fluoro-1-(2-hydroxyethyl)-6-(H-pyrazolo[1,5-a]pyridin-3-yl)-1H-pyrazolo[3,4-b]pyridin-3-yl)benzamide

¹H-NMR (300 MHz, DMSO-d6): 3.97(q, J=5.4 Hz, 2H), 4.56(t, J=5.7 Hz, 2H), 4.96(t, J=5.5 Hz, 1H), 7.17(dd, J=6.8 Hz, 1H), 7.53-7.65(m, 4H), 8.10(d, J=7.3 Hz, 2H), 8.21(d, J=12.2 Hz, 1H), 8.64(d, J=4.3 Hz, 1H), 8.81(d, J=8.8 Hz, 1H), 8.88(d, J=6.9 Hz, 1H), 11.21(s, 1H)

### Prep. Example 1-90

### 2-(3-amino-5-fluoro-6-(5-methoxyH-pyrazolo[1,5-a]pyridin-3-yl)-1H-pyrazolo[3,4-b]pyridin-1-yl)ethanol

¹H-NMR (300 MHz, DMSO-d6): 3.86(q, J=5.7 Hz, 2H), 3.94(s, 3H), 4.30(t, J=6.0 Hz, 2H), 4.60(t, J=5.5 Hz, 2H), 5.14(s, 2H), 6.64(dd, J=2.9 Hz, J=7.5 Hz, 1H), 7.81(d, J=11.6 Hz, 1H), 8.21(d, J=2.8 Hz, 1H), 8.38(d, J=4.1 Hz, 1H), 8.41(d, J=7.6 Hz, 1H)

### Prep. Example 1-91

### 2-(3-benzamido-5-fluoro-6-(5-methoxypyrazolo[1,5-a]pyridin-3-yl)-1H-pyrazolo[3,4-b]pyridin-1-yl)ethyl acetate

¹H-NMR (300 MHz, DMSO-d6): 1.77(s, 3H), 4.05(s, 3H), 4.54(t, J=5.0 Hz, 2H), 4.77(t, J=5.0 Hz, 2H), 6.86(dd, J=2.8 Hz, J=7.5 Hz, 1H), 7.56-7.64(m, 3H), 8.12(d, J=7.1 Hz, 2H), 8.20-8.25(m, 2H), 8.59(d, J=4.5 Hz, 1H), 8.75(d, J=7.5 Hz, 1H), 11.23(s, 1H)

### Prep. Example 1-92

### N-(5-fluoro-1-(2-hydroxyethyl)-6-(5-methoxyH-pyrazolo[1,5-a]pyridin-3-yl)-1H-pyrazolo[3,4-b]pyridin-3-yl)benzamide

¹H-NMR (300 MHz, Methaol-d4): 3.97(s, 3H), 4.05(t, J=5.5 Hz, 2H), 4.58(t, J=5.3 Hz, 2H), 6.65(dd, J=2.8 Hz, J=7.5 Hz, 1H), 7.46-7.56(m, 3H), 7.96(d, J=6.9 Hz, 2H), 8.18(d, J=11.9 Hz, 2H), 8.33(d, J=7.5 Hz, 1H), 8.53(d, J=4.1 Hz, 1H)

### Prep. Example 1-93

### 4-fluoro-N-(5-fluoro-1-(4-fluorobenzoyl)-6-(7-methylH-pyrazolo[1,5-a]pyridin-3-yl)-1H-pyrazolo[3,4-b]pyridin-3-yl)benzamide

¹H-NMR (300 MHz, DMSO-d6): 2.75(s, 3H), 7.07(d, J=6.9 Hz, 1H), 7.37-7.46(m, 5H), 8.09(dd, J=5.6 Hz, J=8.6 Hz, 2H), 8.17(dd, J=5.6 Hz, J=8.6 Hz, 2H), 8.30(d, J=12 Hz, 1H), 8.57(d, J=8.8 Hz, 1H), 8.69(d, J=4.3 Hz, 1H), 11.58(s, 1H)

### Prep. Example 1-94

### N-(1-(cyclopentanecarbonyl)-5-fluoro-6-(H-pyrazolo[1,5-a]pyridin-3-yl)-1H-pyrazolo[3,4-b]pyridin-3-yl)cyclopentanecarboxamide

¹H-NMR (300 MHz, DMSO-d6): 1.56-1.61(m, 2H), 1.65-1.82(m, 8H), 1.87-1.94(m, 4H), 2.01-2.09(m, 4H), 2.95-3.06(m, 2H), 7.17(dd, J=6.8 Hz, 1H), 7.67(dd, J=6.8 Hz, 1H), 8.34(d, J=12.1 Hz, 1H), 8.68(d, J=4.2 Hz, 1H), 8.91(d, J=6.9 Hz, 1H), 9.19(d, J=8.6 Hz, 1H), 11.24(s, 1H)

### Prep. Example 1-95

### 2-(5-fluoro-3-(2-(4-fluorophenyl)acetamido)-6-(H-pyrazolo[1,5-a]pyridin-3-yl)-1H-pyrazolo[3,4-b]pyridin-1-yl)ethyl acetate

¹H-NMR (300 MHz, DMSO-d6): 1.74(s,3H), 3.75(s, 2H), 4.50(t, J=4.8 Hz, 2H), 4.70(t, J=4.7 Hz, 2H), 7.14-7.19(m, 3H), 7.39(dd, J=7 Hz, 2H), 7.58(dd, J=7.3 Hz, 1H), 8.19(d, J=12.5 Hz, 1H), 8.62(d, J=4.1 Hz, 1H), 8.79(d, J=8.9 Hz, 1H), 8.88(d, J=6.8 Hz, 1H), 11.26(s, 1H)

### Prep. Example 1-96

### 2-(5-fluoro-3-(4-fluorobenzamido)-6-(H-pyrazolo[1,5-a]pyridin-3-yl)-1H-pyrazolo[3,4-b]pyridin-1-yl)ethyl acetate

¹H-NMR (300 MHz, CDCl3): 1.83(s, 3H), 4.57(t, J=5.1 Hz, 2H), 4.74(t, J=5.0 Hz, 2H), 6.97(dd, J=6.9 Hz, 1H), 7.18(t, J=8 Hz, 2H), 7.45(dd, J=8.2 Hz, 1H), 7.99(dd, J=5.2 Hz, J=7.5 Hz, 2H), 8.39(t, J=12 Hz, 1H), 8.57-8.60(m, 2H), 8.70(d, J=4.0 Hz, 1H), 8.79(d, J=8.5 Hz, 1H)

### Prep. Example 1-97

### N-(5-fluoro-1-(2-hydroxyethyl)-6-(H-pyrazolo[1,5-a]pyridin-3-yl)-1H-pyrazolo[3,4-b]pyridin-3-yl)cyclopentanecarboxamide

¹H-NMR (300 MHz, DMSO-d6): 1.55-1.57(m, 2H), 1.59-1.74(m, 4H), 1.80-1.90 (m, 2H), 2.89-2.94(m, 1H), 3.90(s, 2H), 4.48(t, J=5.7 Hz, 2H), 7.16(dd, J=6.9 Hz, 1H), 7.59(dd, J=7.2 Hz, 1H), 8.22(d, J=12.4 Hz, 1H), 8.62(d, J=4.3 Hz, 1H), 8.78(d, J=8.9 Hz, 1H), 8.86(d, J=6.9 Hz, 1H), 10.76(s, 1H)

### Prep. Example 1-98

### N-(5-fluoro-1-(2-hydroxyethyl)-6-(H-pyrazolo[1,5-a]pyridin-3-yl)-1H-pyrazolo[3,4-b]pyridin-3-yl)hexanamide

¹H-NMR (300 MHz, DMSO-d6): 0.88(t, J=6.6 Hz, 3H), 1.22-1.35(m, 4H), 1.63 (t, J=7.2 Hz, 2H), 2.39(t, J=7.2 Hz, 2H), 3.90(q, J=5.6 Hz, 2H), 4.48(t, J=5.7 Hz, 2H), 4.90(t, J=5.6 Hz, 1H), 7.16(dd, J=6.7 Hz, 1H), 7.59(dd, J=6.8 Hz, 1H), 8.21(d, J=12.4 Hz, 1H), 8.62(d, J=4.3 Hz, 1H), 8.80(d, J=8.8 Hz, 1H), 8.86(d, J=6.9 Hz, 1H), 10.7(s, 1H)

### Prep. Example 1-99

### N-(5-fluoro-1-(2-hydroxyethyl)-6-(5-methoxyH-pyrazolo[1,5-a]pyridin-3-yl)-1H-pyrazolo[3,4-b]pyridin-3-yl)hexanamide

¹H-NMR (300 MHz, DMSO-d6): 0.89(t, J=6.4 Hz, 3H), 1.29-1.34(m, 4H), 1.64(t, J=7.1 Hz, 2H), 2.39(t, J=7.2 Hz, 2H), 3.38(q, J=5.4 Hz, 2H), 3.99(s, 3H), 4.48(t, J=5.8 Hz, 2H), 4.92(t, J=5.3 Hz, 1H), 6.82(dd, J=2.7 Hz, J=7.5 Hz, 1H), 8.17(d, J=12.4 Hz, 1H), 8.25(d, J=2.7 Hz, 1H), 8.51(d, J=4.3 Hz, 1H), 8.74(d, J=7.5 Hz, 1H), 10.74(s, 1H)

### Prep. Example 1-100

### 2-(5-fluoro-3-(2-methylbenzamido)-6-(H-pyrazolo[1,5-a]pyridin-3-yl)-1H-pyrazolo[3,4-b]pyridin-1-yl)ethyl acetate

¹H-NMR (300 MHz, CDCl₃): 1.82(s, 3H), 3.91 (s, 3H), 4.51-4.60 (m, 2H), 4.73 (t, J=5.4 Hz, 2H), 6.67 (dd, J=2.7, 7.2 Hz, 1H), 7.03(d, J=9.0 Hz, 2H), 7.94 (d, J=9.0 Hz, 2H), 8.19(d, J=3 Hz, 1H), 8.37-8.46 (m, 3H), 8.64(d, J=4.2 Hz, 1H)

### Prep. Example 1-101

### 2-(3-(2,6-difluorobenzamido)-5-fluoro-6-(5-methoxyH-pyrazolo[1,5-a]pyridin-3-yl)-1H-pyrazolo[3,4-b]pyridin-1-yl)ethyl acetate

¹H-NMR (300 MHz, DMSO-d6): 1.73 (s, 3H), 4.02 (s, 3H), 4.51 (t, J=5.1 Hz, 2H), 4.74 (t, J=3.9 Hz, 2H), 6.86 (dd, J=3.0, 7.5 Hz, 1H), 7.27(t, J=8.1 Hz, 2H), 7.59-7.25 (m, 2H), 8.57 (d, J=4.5 Hz, 1H), 8.76 (d, J=7.5 Hz, 1H), 10.12 (bs, 1H), 11.73 (s, 1H)

### Prep. Example 1-102

### 2-(5-fluoro-6-(5-methoxyH-pyrazolo[1,5-a]pyridin-3-yl)-3-(2-methoxybenzamido)-1H-pyrazolo[3,4-b]pyridin-1-yl)ethyl acetate

¹H-NMR (300 MHz, DMSO-d6): 1.73 (s, 3H), 3.97 (s, 3H), 4.04 (s, 3H), 4.51 (bt, 2H), 4.74 (bt, 2H), 6.86 (dd, J=2.4, 7.8 Hz, 1H), 7.12(d, J=6.9 Hz, 1H), 7.24 (bd, 1H), 7.57 (t, J=7.2 Hz, 1H), 7.85 (d, J=8.1 Hz, 1H), 8.23 (d, J=3.0 Hz, 1H), 8.32 (d, J=12.0 Hz, 1H), 8.58 (d, J=4.5 Hz, 1H), 8.76 (d, J=7.5 Hz, 1H), 10.72 (s, 1H)

### Prep. Example 1-103

### 2-(5-fluoro-3-(2-methoxyacetamido)-6-(H-pyrazolo[1,5-a]pyridin-3-yl)-1H-pyrazolo[3,4-b]pyridin-1-yl)ethyl acetate

¹H-NMR (300 MHz, DMSO-d6): 1.76 (s, 3H), 3.40 (s, 3H), 4.12 (s, 2H), 4.49 (t, J=5.4 Hz, 2H), 4.72 (t, J=5.1 Hz, 2H), 7.18 (d, J=6.9 Hz, 1H), 7.59(d, J=7.8 Hz, 1H), 8.23 (d, J=12.3 Hz, 1H), 8.65 (d, J=4.2 Hz, 1H), 8.79 (d, J=9.0 Hz, 1H), 8.89 (d, J=6.9 Hz, 1H), 10.66 (s, 1H)

### Prep. Example 1-104

### 2-(3-(2,5-difluorobenzamido)-5-fluoro-6-(5-methoxyH-pyrazolo[1,5-a]pyridin-3-yl)-1H-pyrazolo[3,4-b]pyridin-1-yl)ethyl acetate

¹H-NMR (300 MHz, DMSO-d6): 1.73 (s, 3H), 4.05 (s, 3H), 4.52 (d, J=5.1 Hz, 2H), 4.75 (d, J=4.8 Hz, 2H), 6.86 (dd, J=2.7, 7.5 Hz, 1H), 7.45-7.49 (m, 2H), 7.49 (bs, 1H), 8.22-8.29 (m, 2H), 8.58 (d, J=4.8 Hz, 1H), 8.78 (d, J=3.6 Hz, 1H), 11.37 (s, 1H)

### Prep. Example 1-105

### 2-(5-fluoro-3-(4-methoxybenzamido)-6-(H-pyrazolo[1,5-a]pyridin-3-yl)-1H-pyrazolo[3,4-b]pyridin-1-yl)ethyl acetate

¹H-NMR (300 MHz, DMSO-d6): 1.86 (s, 3H), 3.90 (s, 3H), 3.97 (q, J=5.8 Hz, 2H), 4.53 (t, J=5.8 Hz, 2H), 4.96 (t, J=5.4H, 1H), 7.07 (d, J=8.7 Hz, 2H), 7.18(t, J=6.6 Hz, 1H), 7.61 (t, J=8.1 Hz, 1H), 8.11 (d, J=8.7 Hz, 2H), 8.21 (d, J=12.0 Hz, 1H), 8.66 (d, J=4.2 Hz, 1H), 8.82 (d, J=9.0 Hz, 1H), 8.89(d, J=6.9 Hz, 1H), 11.06 (s, 1H)

### Prep. Example 1-106

### N-(5-fluoro-1-(2-hydroxyethyl)-6-(H-pyrazolo[1,5-a]pyridin-3-yl)-1H-pyrazolo[3,4-b]pyridin-3-yl)-4-methoxybenzamide

¹H-NMR (300 MHz, DMSO-d6): 3.86 (s, 3H), 3.95 (q, J=6.0 Hz, 2H), 4.55 (t, J=6.0 Hz, 2H), 4.96 (t, J=5.4H, 1H), 7.07 (d, J=8.7 Hz, 2H), 7.18(t, J=6.6 Hz, 1H), 7.61 (t, J=8.1 Hz, 1H), 8.11 (d, J=8.7 Hz, 2H), 8.22 (d, J=12.3 Hz, 1H), 8.64 (d, J=4.8 Hz, 1H), 8.82 (d, J=9.0 Hz, 1H), 8.89(d, J=6.9 Hz, 1H), 11.06 (s, 1H)

### Prep. Example 1-107

### (E)-N-(5-fluoro-1-(2-hydroxyethyl)-6-(H-pyrazolo[1,5-a]pyridin-3-yl)-1H-pyrazolo[3,4-b]pyridin-3-yl)but-2-enamide

¹H-NMR (300 MHz, DMSO-d6): 1.89 (d, J=6.9 Hz, 3H), 3.90 (q, J=5.7 Hz, 2H), 4.50 (t, 1=5.7 Hz, 2H), 4.93 (t, J=6.0 Hz, 1H), 6.27 (d, J=13.8 Hz, 1H), 6.92 (dd, J=6.6, 15 Hz, 1H), 7.17 (t, J=6.9 Hz, 1H), 7,60 (t, J=6.6 Hz, 1H), 8.34 (d, J=12.6 Hz, 1H), 8.64 (d, J=4.2 Hz, 1H), 8.81 (d, J=9.0 Hz, 1H), 8.89 (d, J=6.9 Hz, 1H), 10.94 (s, 1H)

### Prep. Example 1-108

### N-(5-fluoro-1-(2-hydroxyethyl)-6-(H-pyrazolo[1,5-a]pyridin-3-yl)-1H-pyrazolo[3,4-b]pyridin-3-yl)propionamide

¹H-NMR (300 MHz, DMSO-d6): 1.13 (t, J=7.5 Hz, 3H), 2.40-2.48 (m, 2H), 3.90 (q, J=5.1 Hz, 2H), 4.49 (t, J=5.7 Hz, 2H), 4.92 (t, J=5.7 Hz, 1H), 7.17 (t, J=6.0 Hz, 1H), 7,60 (t, J=6.9 Hz, 1H), 8.26 (d, J=12.3 Hz, 1H), 8.64 (d, J=4.2 Hz, 1H), 8.80 (d, J=9.6 Hz, 1H), 8.89 (d, J=6.0 Hz, 1H), 10.77 (s, 1H)

### Prep. Example 1-109

### 2,5-difluoro-N-(5-fluoro-1-(2-hydroxyethyl)-6-(5-methoxyH-pyrazolo[1,5-a]pyridin-3-yl)-1H-pyrazolo[3,4-b]pyridin-3-ynbenzamide

¹H-NMR (300 MHz, DMSO-d6): 3.92-3.93 (bs, 2H), 3.96 (s, 3H), 4.53 (bt, 2H), 4.97 (bs, 1H), 6.84 (bd, 1H), 7.47(bt, 2H), 7.64 (bt, 1H), 8.22-8.27 (m, 2H), 8.54 (bd, 1H), 8.74 (d, J=6.9 Hz, 1H), 11.33 (s, 1H)

### Prep. Example 1-110

### 3-fluoro-N-(5-fluoro-1-(2-hydroxyethyl)-6-(5-methoxyH-pyrazolo[1,5-a]pyridin-3-yl)-1H-pyrazolo[3,4-b]pyridin-3-yl)benzamide

¹H-NMR (300 MHz, DMSO-d6): 3.95 (bs, 2H), 4.02 (s, 3H), 4.56 (bt, 2H), 6.85 (dd, J=3.3, 7.8 Hz, 1H), 7.45-7.48 (m, 1H), 7.59 (m, 1H), 7.96 (d, J=7.5 Hz, 2H), 8.21 (d, J=12.0 Hz, 1H), 8.30 (d, J=2.7 Hz, 1H), 8.55 (d, J=4.5 Hz, 1H), 8.76 (d, J=2.5 Hz, 1H), 11.34 (s, 1H)

### Prep. Example 1-111

### N-(5-fluoro-1-(2-hydroxyethyl)-6-(H-pyrazolo[1,5-a]pyridin-3-yl)-1H-pyrazolo[3,4-b]pyridin-3-yl)-2-methoxyacetamide

¹H-NMR (300 MHz, DMSO-d6): 3.40 (s, 3H), 3.89 (m, 2H), 4.12 (s, 2H), 4.50 (t, J=6.0 Hz, 2H), 4.94 (bs, 1H), 7.17 (t, J=6.9 Hz, 1H), 7.61 (t, J=6.9 Hz, 1H), 8.22 (d, J=12.3 Hz, 1H), 8.64 (d, J=4.2 Hz, 1H), 8.80 (d, J=9.0 Hz, 1H), 8.89 (d, J=6.9 Hz, 1H), 10.63 (s, 1H)

### Prep. Example 1-112

### N-(5-fluoro-1-(2-hydroxyethyl)-6-(H-pyrazolo[1,5-a]pyridin-3-yl)-1H-pyrazolo[3,4-b]pyridin-3-yl)-2-methylbenzamide

¹H-NMR (300 MHz, DMSO-d6): 2.47 (s, 3H), 3.94 (q, J=5.4 Hz, 2H), 4.54 (t, J=5.4 Hz, 2H), 4.95 (t, J=5.4H, 1H), 7.17(t, J=6.9 Hz, 1H), 7.28-7.34 (m, 2H), 7.39-7.43 (m, 1H), 7.58-7.66 (m, 2H), 8.26 (d, J=12.3 Hz, 1H), 8.65 (d, J=4.5 Hz, 1H), 8.82 (d, J=9.0 Hz, 1H), 8.89 (d, J=6.9 Hz, 1H), 11.09 (s, 1H)

### Prep. Example 1-113

### 2-(5-fluoro-3-propionamido-6-(H-pyrazolo[1,5-a]pyridin-3-yl)-1H-pyrazolo[3,4-b]pyridin-1-yl)ethyl acetate

¹H-NMR (300 MHz, DMSO-d6): 1.13 (t, J=7.5 Hz, 3H), 2.40-2.48 (m, 2H), 3.90 (q, J=5.1 Hz, 2H), 4.49 (t, J=5.7 Hz, 2H), 4.92 (t, J=5.7 Hz, 1H), 7.17 (t, J=6.0 Hz, 1H), 7.60 (t, J=6.9 Hz, 1H), 8.26 (d, J=12.3 Hz, 1H), 8.64 (d, J=4.2 Hz, 1H), 8.80 (d, J=9.6 Hz, 1H), 8.80 (d, J=6.0 Hz, 1H), 10.77 (s, 1H)

### Prep. Example 1-114

### 2-(5-fluoro-3-(2-fluorobenzamido)-6-(H-pyrazolo[1,5-a]pyridin-3-yl)-1H-pyrazolo[3,4-b]pyridin-1-yl)ethyl acetate

¹H-NMR (300 MHz, DMSO-d6): 1.78 (s, 3H), 4.52 (t, J=4.8 Hz, 2H), 4.75 (t, J=5.1 Hz, 2H), 7.18 (t, J=6.9 Hz, 1H), 7,33-7.40 (m, 2H), 7.58-7.65 (m, 2H), 7.79 (t, J=7.5 Hz, 1H), 8.29 (d, J=12.3 Hz, 1H), 8.67 (d, J=4.2 Hz, 1H), 8.81 (d, J=9.0 Hz, 1H), 8.90 (d, J=6.9 Hz, 1H), 11.27 (s, 1H)

### Prep. Example 1-115

### 2-cyclohexyl-N-(1-(2-cyclohexylacetyl)-5-fluoro-6-(H-pyrazolo[1,5-a]pyridin-3-yl)-1H-pyrazolo[3,4-b]pyridin-3-yl)acetamide

¹H-NMR (300 MHz, DMSO-d6): 0.85-1.09 (m, 5H), 1.14-1.32 (m, 6H), 1.62-1.85 (m, 11H), 1.96 (d, J=6.9 Hz, 2H), 2.29 (d, J=6.9 Hz, 1H), 7.15 (t, J=6.6 Hz, 1H), 7.58 (t, J=7.8 Hz, 1H), 8.20 (d, J=12.3 Hz, 1H), 8.61 (d, J=4.2 Hz, 1H), 8.73 (d, J=9.0 Hz, 1H), 8.87 (d, J=6.9 Hz, 1H), 13.16 (s, 1H)

A description is also given of the synthesis examples based on Reaction Scheme 2, below.

### Synthesis Example 18

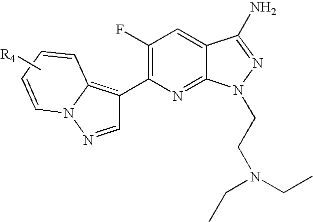

wherein R₄ is as defined as in Chemical Formula 1.

To a solution of the derivative obtained in Synthesis Example 10 serving as a starting material in DMF, were added 2-(diethylamino)ethylamine (1.6775 mmol) and Cs₂CO₃ (3.3552 mmol), followed by refluxing for 16 hours. The organic material was extracted using excess amount of water and ethyl acetate. The organic phase was dried over Na₂SO₄ and concentrated under vacuum. Purification through column chromatography using methanol/methylene chloride afforded the desired product. Typical examples of the products are given in Table 4, below.

### Synthesis Example 19

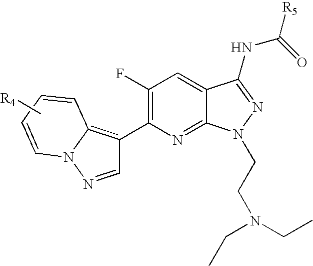

wherein R₄ and R₅ are as defined as in Chemical Formula 1.

To a solution of the derivative obtained in Synthesis Example 18 serving as a starting material in THF, were added DIEA (0.3538 mmol) and acyl chloride (0.2299 mmol), followed by stirring at room temperature for 4 hours. The addition of 1N HCl formed a precipitate which was then filtered and washed with ethyl acetate. This filtrate was compressed and dried under vacuum to afford the desired product as a yellow solid. Typical examples of the product are given in Table 4, below.

Typical examples of the compounds represented by Chemical Formula 2, prepared according to Reaction Scheme 2, are summarized in Table 4, below. In Table 4, M stands for molecular weight, and M+H represents mass spectrum values measured using mass spectrophotometer (ESI-MS).

TABLE 4

| Prep. | | | |
|---|---|---|---|
| Example No. | Molecular Structure | [M] | [M + H]+ |
| 1-116 | 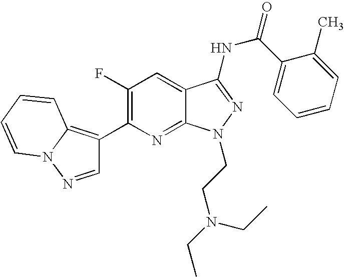 | 485 | 486 |
| 1-117 | 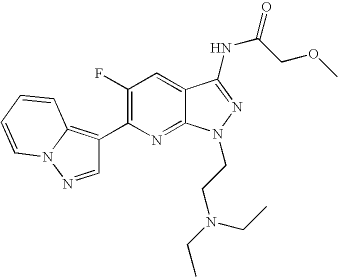 | 439 | 440 |

The preparation examples of the above compounds are described below in detail:

### Prep. Example 1-116

### N-(1-(2-(diethylamino)ethyl)-5-fluoro-6-(H-pyrazolo[1,5-a]pyridin-3-yl)-1H-pyrazolo[3,4-b]pyridin-3-yl)-2-methylbenzamide

¹H-NMR (300 MHz, DMSO-d6): 0.88 (t, J=7.2 Hz, 6H), 2.46 (s, 3H), 2.52-2.47 (m, 4H), 2.91 (t, J=6.6 Hz, 2H), 4.54 (t, J=6.6 Hz, 2H), 7.17 (t, J=6.9 Hz, 1H), 7.28-7.33 (m, 2H), 7.41 (t, J=7.2 Hz, 1H), 7.55-7.60 (m, 2H), 8.25 (d, J=12.0 Hz, 1H), 8.65 (d, J=4.2 Hz, 1H), 8.40 (d, J=8.7 Hz, 1H), 8.89 (d, J=6.9 Hz, 1H), 11.08 (s, 1H)

### Prep. Example 1-117

### N-(1-(2-(diethylamino)ethyl)-5-fluoro-6-(H-pyrazolo[1,5-a]pyridin-3-yl)-1H-pyrazolo[3,4-b]pyridin-3-yl)-2-methoxyacetamide

¹H-NMR (300 MHz, DMSO-d6): 1.19 (bs, 6H), 3.24 (bs, 4H), 3.41 (s, 3H), 3.57-3.62 (m, 2H), 4.14 (s, 2H), 4.93 (bs, 2H), 7.19 (t, J=7.5 Hz, 1H), 7.62 (t, J=8.4 Hz, 1H), 8.27 (d, J=12.0 Hz, 1H), 8.66 (d, J=4.2 Hz, 1H), 8.90 (d, J=7.5 Hz, 2H), 10.67 (s, 1H)

Further synthesis examples according to Reaction Scheme are given as follows.

### Synthesis Example 20

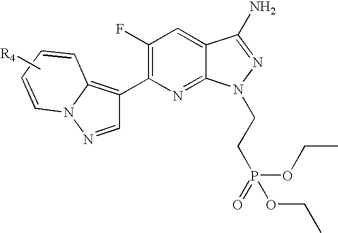

wherein R₄ is as defined as in Chemical Formula 1.

To a solution of the derivative obtained in Synthesis Example 10 in DMF was added Cs₂CO₃ (4.6598 mmol), and the reaction mixture was left at room temperature for 30 min for reaction. To this mixture was slowly added bromo ethylphosphonate (2.1435 mmol), followed by stirring at room temperature for 18 hours. Extraction with ethyl acetate and water gave an organic phase which was then dried over Na₂SO₄ and concentrated under vacuum. Purification through column chromatography using methanol/methylene chloride afforded the desired product. Examples of the product are given in Table 5, below.

### Synthesis Example 21

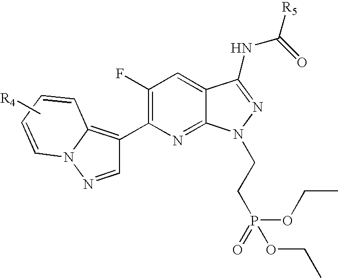

wherein R₄ and R₅ are as defined as in Chemical Formula 1.

To a stirred solution of the derivative obtained in Synthesis Example 20 serving as a starting material in THF, were added DIEA (0.3516 mmol) and acyl chloride (0.2285 mmol), followed by stirring at room temperature for 4 hours. The addition of 1N HCl gave a precipitate which was then filtered and washed with ethyl acetate. The filtrate was compressed and dried under vacuum to afford the desired product as a yellow solid. Typical examples of the product are given in Table 5, below.

Typical examples of the compounds represented by Chemical Formula 2, prepared according to Reaction Scheme 2, are described in Table 5, below. In Table 5, M stands for molecular weight, and M+H represents mass spectrum values measured using a mass spectrophotometer (ESI-MS).

TABLE 5

| Prep. | | | |
|---|---|---|---|
| Example No. | Molecular Structure | [M] | [M + H]+ |
| 1-118 | 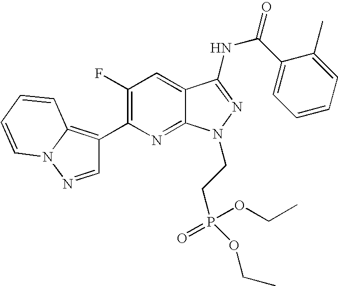 | 550 | 551 |
| 1-119 | 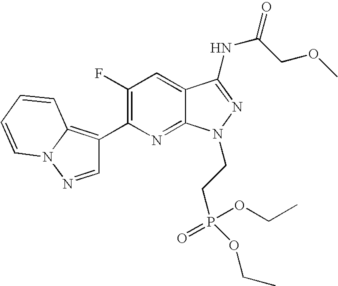 | 504 | 505 |
| 1-120 | 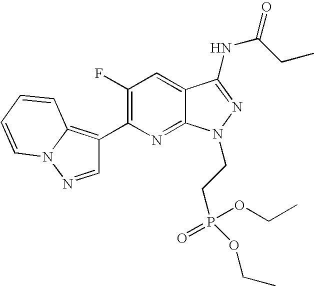 | 488 | 489 |
| 1-121 | 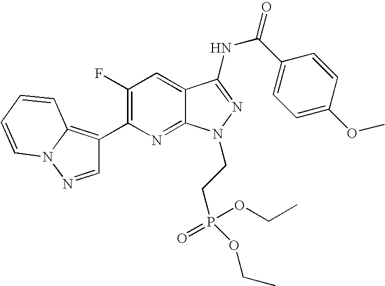 | 566 | 567 |
| 1-122 | 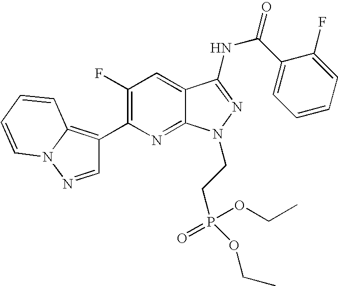 | 554 | 555 |

The preparation examples of the above compounds are described below in detail:

### Prep. Example 1-118

### diethyl 2-(5-fluoro-3-(2-methylbenzamido)-6-(H-pyrazolo[1,5-a]pyridin-3-yl)-1H-pyrazolo[3,4-b]pyridin-1-yl)ethylphosphonate

¹H-NMR (300 MHz, DMSO-d6): 1.30 (t, J=6.9 Hz, 6H), 2.42-2.54 (m, 2H), 2.59 (s, 3H), 4.13 (m, 4H), 4.79 (m, 2H), 6.98 (t, J=9.6 Hz, 1H), 7,32 (d, J=10.5 Hz, 2H), 7.42 (m, 2H), 7.62 (d, J=7.2 Hz, 1H), 8.22 (s, 1H), 8.44 (d, J=12.3 Hz, 1H), 8.59 (d, J=6.9 Hz, 1H), 8.72 (d, J=4.2 Hz, 1H), 8.87(d, J=8.7 Hz, 1H)

### Prep. Example 1-119

### diethyl 2-(5-fluoro-3-(2-methoxyacetamido)-6-(H-pyrazolo[1,5-a]pyridin-3-yl)-1H-pyrazolo[3,4-b]pyridin-1-yl)ethylphosphonate

¹H-NMR (300 MHz, DMSO-d6): 1.29 (t, J=6.9 Hz, 6H), 2.41-2.53 (m, 2H), 3.55 (s, 3H), 4.06-4.16 (m, 6H), 4.73-4.81 (m, 2H), 6.97 (t, J=5.4 Hz, 1H), 7,42 (t, J=6.9 Hz, 1H), 8.38 (d, J=12.3 Hz, 1H), 8.58 (d, J=6.9 Hz, 1H), 8.70 (d, J=4.2 Hz, 1H), 8.85(d, J=9.0 Hz, 1H), 8.90 (bs, 1H)

### Prep. Example 1-120

### diethyl 2-(5-fluoro-3-propionamido-6-(H-pyrazolo[1,5-a]pyridin-3-yl)-1H-pyrazolo[3,4-b]pyridin-1-yl)ethylphosphonate

¹H-NMR (300 MHz, DMSO-d6): 1.27-1.33 (m, 9H), 2.40-2.55 (m, 4H), 4.06-4.15 (m, 4H), 4.75 (q, J=6.0 Hz, 2H), 6.97 (t, J=5.7 Hz, 1H), 7,41 (t, J=6.9 Hz, 1H), 8.06 (s, 1H), 8.34 (d, J=12.0 Hz, 1H), 8.58 (d, J=6.9 Hz, 1H), 8.69 (d, J=4.2 Hz, 1H), 8.84 (d, J=9.0 Hz, 1H)

### Prep. Example 1-121

### diethyl 2-(5-fluoro-3-(4-methoxybenzamido)-6-(H-pyrazolo[1,5-a]pyridin-3-yl)-1H-pyrazolo[3,4-b]pyridin-1-yl)ethylphosphonate

¹H-NMR (300 MHz, DMSO-d6): 1.30 (t, J=6.9 Hz, 6H), 2.43-2.54 (m, 2H), 3.90 (s, 3H), 4.11 (m, 4H), 4.72-4.83 (m, 2H), 6.96-7.04 (m, 3H), 7,42 (t, J=8.1 Hz, 1H), 7.94 (d, J=8.7 Hz, 1H), 8.44 (d, J=12.0 Hz, 1H), 8.54 (s, 1H), 8.59 (d, J=6.9 Hz, 1H), 8.71 (d, J=4.2 Hz, 1H), 8.86 (d, J=8.7 Hz, 1H)

### Prep. Example 1-122

### diethyl 2-(5-fluoro-3-(2-fluorobenzamido)-6-(H-pyrazolo[1,5-a]pyridin-3-yl)-1H-pyrazolo[3,4-b]pyridin-1-yl)ethylphosphonate

¹H-NMR (300 MHz, DMSO-d6): 1.29 (t, J=7.2 Hz, 6H), 2.45-2.59 (m, 2H), 4.12 (m, 4H), 4.76-4.85 (m, 2H), 6.90-7.05 (m, 2H), 7.34-7.46 (m, 2H), 7.56-7.62 (m, 1H), 8.26 (t, J=7.8 Hz, 1H), 8.43 (d, J=12.0 Hz, 1H), 8.59 (d, J=6.9 Hz, 1H), 8.72 (d, J=4.2 Hz, 1H), 8.93 (d, J=3.9 Hz, 1H).

The following 6-(H-pyrazolo[1,5-a]pyridin-3-yl)-1H-pyrazolo[3,4-b]pyridine derivatives are used as starting materials in the fifth step of Reaction Scheme 4. A detailed description is given of the preparation of the 6-(H-pyrazolo[1,5-a]pyridin-3-yl)-1H-pyrazolo[3,4-b]pyridine derivatives.

### I. Synthesis of 6-(H-pyrazolo[1,5-a]pyridin-3-yl)-1H-pyrazolo[3,4-b]pyridine Derivative

### Synthesis Example 22

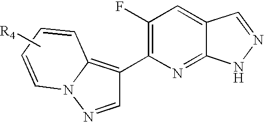

wherein R₄ is as defined as in Chemical Formula 1.

To a stirred solution of the 5-fluoro-6-pyrazole[1,5-a]pyridin-3-yl-1H-pyrazolo[3,4-b]pyridin-3-yl-amine derivative (0.01 mol) obtained in Synthesis Example 10, serving as a starting material, was added isoamyl nitrite (0.012 mol), followed by refluxing for 5 hours. After checking the progression of the reaction, methylene chloride and water were added to terminate the reaction. The organic phase thus formed was dried over Na₂SO₄ and concentrated under vacuum. Column chromatography using MeOH/MC afforded the desired product. Typical examples of the product are given in Table 6, below.

### Synthesis Example 23

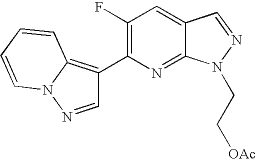

The 5-fluoro-6-(H-pyrazolo[1,5-a]pyridin-3-yl)-1H-pyrazolo[3,4-b]pyridine (1 mmol) obtained in Synthesis Example 11, serving as a starting material, was sufficiently dissolved in DMF and then added Cs₂CO₃ (3 mmol). The mixture was stirred at room temperature for 30 min. Then, 2-bromoethylacetate (1.2 mol) was added, followed by heating at 50° C. for 18 hours.

After confirming that the reaction was completed, the reaction mixture was extracted with ethyl acetate, and washed with water. The organic phase thus formed was dried over Na₂SO₄ and concentrated in vacuo. Column chromatography using ethyl acetate/hexane afforded the desired product (yield: 25%).

¹H-NMR (300 MHz, DMSO-d6): δ 8.88(d, J=6.8 Hz, 1H), 8.81(d, J=8.9 Hz, 1H), 8.51(d, J=4.4 Hz, 1H), 8.18(d, J=11.69 Hz, 1H), 8.12(s, 1H), 7.59(dd, J=5.1 Hz, 1H), 7.17(dd, J=5.1 Hz, 1H), 4.79(dd, J=5.2 Hz, 2H), 4.52(dd, J=5.2 Hz, 2H), 1.75(s, 3H)

### Synthesis Example 24

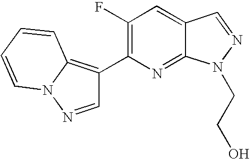

To a stirred solution of 2-(5-fluoro-6-(H-pyrazolo[1,5-a]pyridin-3-yl)-1H-pyrazolo[3,4-b]pyridin-1-yl)ethyl acetate (1 mmol), obtained in Synthesis Example 23, serving as a starting material, in MeOH was added K₂CO₃ (3 mmol), followed by stirring at room temperature for 1 hour. After the removal of the solvent under vacuum, the residue was extracted with MC. The organic phase thus formed was dried over Na₂SO₄ and concentrated under vacuum. Column chromatography using ethyl acetate/hexane afforded the desired product (yield: 17%).

¹H-NMR (300 MHz, DMSO-d6): δ 8.88(d, J=6.8 Hz, 1H), 8.81(d, J=8.9 Hz, 1H), 8.6(d, J=4.3 Hz, 1H), 8.17(d, J=11.67 Hz,1H), 8.10(s, 1H), 7.16(dd, J=6.9 Hz, 1H), 4.91(dd, J=5.65 Hz, 1H), 4.58(dd, J=5.7 Hz, 2H), 3.94(q, J=5.5 Hz, 2H)

### Synthesis Example 25

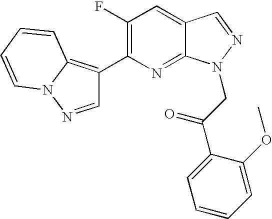

The 5-fluoro-6-(H-pyrazolo[1,5-a]pyridin-3-yl)-1H-pyrazolo[3,4-b]pyridine (1 mmol), obtained in Synthesis Example 11, serving as a starting material, was sufficiently dissolved in DMF and mixed with Cs₂CO₃ (3 mmol) and stirred at room temperature for 30 min. Subsequently, 2-bromo-2′-methoxyacetophenone (1.2 mmol) was added to the reaction mixture which was then heated overnight at 50° C. After confirming that the reaction is completed, the reaction mixture was extracted with ethyl acetate and washed with water. The organic phase thus formed was dried over Na₂SO₄ and concentrated under vacuum. MPLC afforded the desired product (yield: 12%).

¹H-NMR (300 MHz, DMSO-d6): δ 8.85(d, J=6.56 Hz, 1H), 8.62(d, J=4.27 Hz, 1H), 8.48(d, J=8.88 Hz, 1H), 8.22(d, J=11.56 Hz, 1H), 8.16(s, 1H) 7.69(m, 1H), 7.06-7.33(aromatic proton, 5H), 5.99(bs, 2H), 4.03(s, 3H)

### Synthesis Example 26

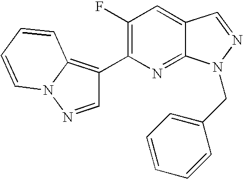

The 5-fluoro-6-(H-pyrazolo[1,5-a]pyridin-3-yl)-1H-pyrazolo[3,4-b]pyridine (1 mmol) obtained in Synthesis Example 11, serving as a starting material, was sufficiently dissolved in DMF and mixed with Cs₂CO₃ (3 mmol) and stirred at room temperature for 30 min. The addition of benzyl bromide (1.2 mmol) was followed by heating overnight at 50° C. After confirming that the reaction is completed, the reaction mixture was extracted with ethyl acetate and washed with water. The organic phase thus formed was concentrated and the concentrate was purified through recrystallization in a suitable solvent to afford the desired product (yield: 15%).

¹H-NMR (300 MHz, DMSO-d6): δ 8.85(d, J=6.9 Hz, 1H), 8.79(d, J=8.6 Hz, 1H), 8.59(d, J=4.6 Hz, 1H), 8.52(s, 1H), 8.1(d, J=12.06 Hz, 1H), 7.55(dd, J=8.2, 7.81 Hz, 1H), 7.38(m, 4H), 7.34(m, 1H), 7.14(ddd, J=6.7, 1 Hz, 1H), 5.6(bs, 2H)

Typical examples of the compounds represented by Chemical Formula 3, prepared according to Reaction Scheme 4, are summarized in Table 6, below. In Table 6, M stands for molecular weight, and M+H represents mass spectrum values measured using a mass spectrophotometer (ESI-MS).

TABLE 6

| Prep. | | | |
|---|---|---|---|
| Example No. | Molecular Structure | [M] | [M + H]+ |
| 1-123 | 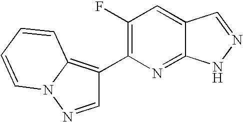 | 253 | 254 |
| 1-124 | 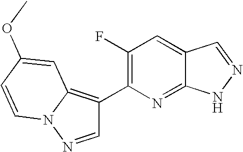 | 283 | 284 |
| 1-125 | 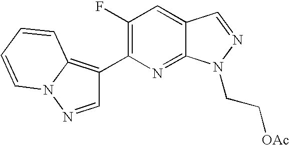 | 339 | 340 |
| 1-126 | 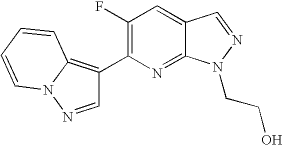 | 297 | 298 |
| 1-127 | 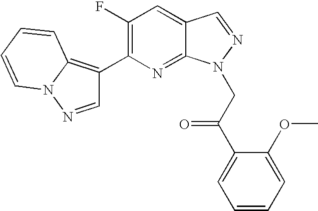 | 401 | 402 |
| 1-128 | 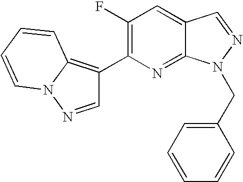 | 343 | 344 |

The preparation examples of the above compounds are described below in detail:

### Prep. Example 1-123

### 5-fluoro-6-(H-pyrazolo[1,5-a]pyridin-3-yl)-1H-pyrazolo[3,4-b]pyridine

¹H-NMR (300 MHz, DMSO-d6): 7.15(ddd, J=6.8, 1.3 Hz, 1H), 7.58(m, 1H), 8.1(s, 1H), 8.17(d, J=8.6 Hz, 1H), 8.61(d, J=4.3 Hz, 1H), 8.75(d, J=8.9 Hz, 1H), 8.87(d, J=6.9 Hz, 1H), 13.64(s,1H)

### Prep. Example 1-124

### 5-fluoro-6-(5-methoxyH-pyrazolo[1,5-a]pyridin-3-yl)-1H-pyrazolo[3,4-b]pyridine

¹H-NMR (300 MHz, DMSO-d6): 3.99(s, 3H), 8.07 (s, 1H), 8.12 (d, J=11.9 Hz), 8.21(d, J=2.6 Hz, 1H), 8.51(d, J=4.6 Hz, 1H), 8.73 (d, J=7.58 Hz, 1H), 13.61(s, 1H)

### Prep. Example 1-125

### 2-(5-fluoro-6-(H-pyrazolo[1,5-a]pyridin-3-yl)-1H-pyrazolo[3,4-b]pyridin-1-yl)ethyl acetate

¹H-NMR (300 MHz, DMSO-d6): 1.75(s, 3H), 4.52(dd, J=5.2 Hz, 2H), 4.79 (dd, J=5.2 Hz, 2H), 7.17(dd, J=5.1 Hz, 1H), 7.59 (dd, J=5.1 Hz, 1H), 8.12(s, 1H), 8.18(d, J=11.69 Hz, 1H), 8.51(d, J=4.4 Hz, 1H), 8.81(d, J=8.9 Hz, 1H), 8.88(d, J=6.8 Hz, 1H)

### Prep. Example 1-126

### 2-(5-fluoro-6-(H-pyrazolo[1,5-a]pyridin-3-yl)-1H-pyrazolo[3,4-b]pyridin-1-yl)ethanol

¹H-NMR (300 MHz, DMSO-d6): 3.94(q, J=5.5 Hz, 2H), 4.58(dd, J=5.7 Hz, 2H), 4.91(dd, J=5.65 Hz, 1H), 7.16(dd, J=6.9 Hz, 1H), 8.10(s, 1H), 8.17(d, J=11.67 Hz,1H), 8.6(d, J=4.3 Hz, 1H), 8.81(d, J=8.9 Hz, 1H), 8.88(d, J=6.8 Hz, 1H)

### Prep. Example 1-127

### 2-(5-fluoro-6-(H-pyrazolo[1,5-a]pyridin-3-yl)-1H-pyrazolo[3,4-bripyridin-1-yl)-1-(2-methoxyphenyl)ethanone

¹H-NMR (300 MHz, DMSO-d6): 4.03(s, 3H), 5.99(bs, 2H), 7.06-7.33 (m, 5H), 7.69(m, 1H), 8.16(s, 1H), 8.22(d, J=11.56 Hz, 1H), 8.48(d, J=8.88 Hz, 1H), 8.62(d, J=4.27 Hz, 1H), 8.85(d, J=6.56 Hz, 1H)

### Prep. Example 1-128

### 1-benzyl-5-fluoro-6-(H-pyrazolo[1,5-a]pyridin-3-yl)-1H-pyrazolo[3,4-b]pyridine

¹H-NMR (300 MHz, DMSO-d6): 5.6(bs, 2H), 7.14(ddd, J=6.7, 1 Hz, 1H), 7.34(m, 1H), 7.38(m, 4H), 7.55(dd, J=8.2, 7.81 Hz, 1H), 8.1(d, J=12.06 Hz, 1H), 8.52(s, 1H), 8.59(d, J=4.6 Hz, 1H), 8.79(d, J=8.6 Hz, 1H), 8.85(d, J=6.9 Hz, 1H)

3. Pharmaceutical Composition

The present invention provides a pharmaceutical composition comprising a compound represented by Chemical Formula 1 or a pharmaceutically acceptable salt thereof as an active ingredient in combination with one or more inactive carriers or diluents. The pharmaceutical composition of the present invention can be used to treat diabetes, Alzheimer's disease, CNS disorders and hypertrophic cardiomyopathy. The compounds and pharmaceutically acceptable salts thereof in accordance with the present invention are effective as an active ingredient inhibitory of protein kinases, especially GSK-3 in warm-blooded animals.

### Test Example 1

### Assay for Inhibitory Activity Against GSK3β

The compounds of the present invention were assayed for inhibitory activity against GSK3β. The GSK-3 is implicated in the incidence of various diseases including diabetes, Alzheimer's disease, CNS disorders and hypertrophic cardiomyopathy.

GSK3β was purified from Sf21 cells using a procedure described in the literature, R. Dajani et. al. (Cell 2001, 195, 721-732). GSK3β activity was examined at room temperature in 50 μl of 40 mM Tris-HCl, pH7.4, 10 mM MgCl₂, 1 mM DTT, 0.2 mM EDTA, 200 μM NaVO₃, 10 mM β-glyceralphosphate, 1 mM EGTA buffer containing 750 nM ATP and 4 μM GSY-2 phosphopeptide (substrate). After incubation at room temperature for 30 mM, Kinase-Glo reagent (Promega) was added to reaction mixtures of Preparation Examples 1-1 to 1-12, 1-80 to 1-90, and 1-92. The well plates were incubated at room temperature for 10 mM to stabilize luminescent signals. The luminescent intensity, reflecting the quantity of ATP, was recorded using a counter (Wallac Victor 1420 multilabel counter) and used to determine % inhibition of GSK3β as compared to ATP level in the control to which none of the compounds of the present invention were added. The results are summarized in Table 7, below.

TABLE 7

| | | GSK3β | GSK3β | IC50 of |
|---|---|---|---|---|
| Preparation | | inhibition % | inhibition % | GSK3β |
| Example | F.W. | at 25 μM | at 50 μM | (μM) |
| 1-1 | 336.32 | 80.8 | 96.3 | 10.18 |
| 1-2 | 364.38 | 76.9 | 95.3 | 14.94 |
| 1-3 | 350.35 | 11.6 | 53.1 | — |
| 1-4 | 378.40 | 63.8 | 82.1 | — |
| 1-5 | 411.39 | 35.1 | 78.0 | — |
| 1-6 | 418.40 | 48.3 | 70.8 | — |
| 1-7 | 429.45 | 71.2 | 92.3 | 13.32 |
| 1-8 | 366.35 | 37.7 | 61.6 | — |
| 1-9 | 434.40 | 58.2 | 94.6 | 20.01 |
| 1-10 | 404.37 | 77.4 | 90.7 | 14.44 |
| 1-11 | 268.25 | 30.0 | — | — |
| 1-12 | 298.28 | 87.7 | — | — |
| 1-80 | 368.36 | 19.7 | 37.8 | — |
| 1-81 | 490.46 | 81.9 | 97.5 | NA |
| 1-82 | 354.34 | 64.5 | 80.4 | — |
| 1-83 | 384.36 | 81.2 | 87.2 | — |
| 1-84 | 422.41 | 24.4 | 49.5 | — |
| 1-85 | 450.47 | 68.6 | 90.1 | 34.86 |
| 1-86 | 448.42 | 47.9 | 86.5 | — |
| 1-87 | 312.3 | 26.7 | — | — |
| 1-88 | 458.44 | 23.0 | — | — |
| 1-89 | 416.41 | 1.5 | — | — |
| 1-90 | 342.33 | 115.5 | — | — |
| 1-92 | 446.43 | 116.7 | — | — |
| 1-93 | 526.47 | 51.6 | 88.6 | — |
| 1-94 | 460.50 | 41.7 | 87.2 | — |
| 1-95 | 490.46 | 53.2 | 78.9 | — |

Taken together, the data obtained from the experiments demonstrate that the compounds of the present invention have high inhibitory activity against GSK3β. Accordingly, the compounds represented by Chemical Formula 1 or pharmaceutically acceptable salts thereof can act as inhibitors against protein kinases, especially GSK-3 and can be used for the prevention and treatment of various diseases associated with GSK-3 activation, including diabetes, Alzheimer's disease, CNS disorders and hypertrophic cardiomyopathy.

1. A compound having the following Chemical Formula I: 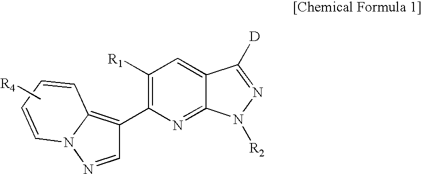
wherein:
D is hydrogen or —NR₃R₃′;
R₁ is hydrogen, a straight or branched C₁˜C₈ alkyl, a C₁˜C₈ alkoxy, or halogen;
R₂, R₃, and R₃′ are each independently hydrogen, a straight or branched C₁˜C₈ alkyl, or —(X₁)—R₅;
wherein R₃ and R₃′ may be combined to each other to form a 6-membered heterocycloalkyl which is unsubstituted or substituted with a straight or branched C₁˜C₈ alkyl and contains one or more heteroatoms selected from N and O,
X₁ is a straight or branched C₁˜C₈ alkylene, —O—, —CO—, 13 (CO)₂13 , —(SO)—, —(SO₂)—, —CH₂(C═O)—, —C(═O)CH₂—, or a single bond; and
R₅ is:
hydroxy; carboxy;
a straight or branched C₁˜C₈ alkyl;
a straight or branched C₁˜C₈ alkyl substituted with a C₂˜C₈ dialkylamino;
a straight or branched C₁˜C₈ alkyl substituted with a C₆˜C₂₀ aryl;
a straight or branched C₁˜C₈ alkyl substituted with a halogen-substituted C₆˜C₂₀ aryl;
a straight or branched C₁˜C₈ alkyl substituted with a C₃˜C₈ heterocycloalkyl containing one or more heteroatoms selected from N and O;
a straight or branched C₁˜C₈ alkyl substituted with a C₃˜C₈ heterocycloalkyl which is substituted with a straight or branched C₁˜C₈ alkyl and contains one or more heteroatoms selected from N and O;
a C₃˜C₈ cycloalkyl; a straight or branched C₁˜C₈ hydroxyalkyl;
a C₁˜C₈ alkoxy;
a C₁˜C₈ acetoxy;
a C₂˜C₈ alkenyl;
nitryl;
a C₂˜C₈ alkenyl substituted with a C₆˜C₂₀ aryl;
a C₂˜C₈ alkenyl substituted with a halogen-substituted C₆˜C₂₀ aryl;
a C₂˜C₈ alkynyl;
a C₂˜C₈ alkynyl substituted with a C₆˜C₂₀ aryl;
a C₂˜C₈ alkynyl substituted with a halogen-substituted C₆˜C₂₀ aryl;
a C₆˜C₂₀ aryl;
a C₆˜C₂₀ aryl substituted with one or more substituents selected from halogen, cyano, a straight or branched C₁˜C₈ alkyl, CF₃, amino, SO₂ and a C₁˜C₈ alkoxy;
a C₆˜C₂₀ aryl substituted with a C₃˜C₈ heterocycloalkyl containing one or more heteroatoms selected from N and O;
a C₆˜C₂₀ aryl substituted with a C₃˜C₈ heterocycloalkyl which is substituted with a straight or branched C₁˜C₈ alkyl and contains one or more heteroatoms selected from N and O;
a C₆˜C₂₀ aryl substituted with a straight or branched C₁˜C₈ alkyl substituted with a C₃˜C₈ heterocycloalkyl containing one or more heteroatoms selected from N and O;
a C₆˜C₂₀ aryl substituted with a straight or branched C₁˜C₈ alkyl substituted with a C₃˜C₈ hetrocycloalkyl which is substituted with a straight or branched C₁˜C₈alkyl and contains one or more heteroatoms selected from N and O;
a C₆˜C₂₀ aryl substituted with a C₂˜C₈ dialkylamino;
a C₃˜C₈ heterocycloalkyl containing one or more heteroatoms selected from N and O;
a C₃˜C₈ heterocycloalkyl which is unsubstituted or substituted with halogen and contains one or more heteroatoms selected from N and O;
phosphonate;
phosphonate which is unsubsitituted or substituted with a straight or branched C₁˜C₈ alkyl; or
NA₁A₂, wherein, A₁ or A₂ may be the same or different and are each independently
hydrogen;
a straight or branched C₁˜C₈ alkyl;
a straight or branched C₁˜C₈ alkyl which is unsubstituted or substituted with phenyl;
a C₂˜C₈ alkenyl;
a C₆˜C₂₀ aryl;
a halogen-substituted C₆˜C₂₀ aryl;
a C₆˜C₂₀ aryl substituted with a straight or branched C₁˜C₄ alkyl; or
a C₆˜C₂₀ aryl substituted with a C₁˜C₄ alkoxy; and
R₄ is hydrogen, hydroxy, halogen, a C₂˜C₈ dialkylamino, or —(X₂)—R₆;
wherein,
X₂ is a straight or branched C₁˜C₈ alkylene, a C₂˜C₈ alkenylene, a C₆˜C₂₀ arylene, a single bond, CO or SO₂, and
R₆ is:
a straight or branched C₁˜C₈ alkyl;
a straight or branched C₁˜C₈ alkyl substituted with a C₆˜C₂₀ aryl;
a straight or branched C₁˜C₈ alkyl substituted with a halogen-substituted C₆˜C₂₀ aryl;
a C₃˜C₈ cycloalkyl;
a C₁˜C₈ alkoxy;
a C₂˜C₈ alkenyl;
a C₂˜C₈ alkenyl substituted with a C₆˜C₂₀ aryl;
a C₂˜C₈ alkenyl substituted with a C₆˜C₂₀ aryl substituted with a C₁˜C₈ alkoxy;
a C₂˜C₈ alkenyl substituted with a halogen-substituted C₆˜C₂₀ aryl;
a C₂˜C₈ alkynyl;
a C₂˜C₈ alkynyl substituted with a C₆˜C₂₀ aryl;
a C₂˜C₈ alkynyl substituted with a halogen-substituted C₆˜C₂₀ aryl;
a C₆˜C₂₀ aryl;
a C₆˜C₂₀ aryl substituted with a C₁˜C₈ alkoxy;
a C₆˜C₂₀ aryl substituted with a straight or branched C₁˜C₈ alkyl;
a halogen-substituted C₆˜C₂₀ aryl;
a C₆˜C₂₀ aryl substituted with a halogen-substituted, straight or branched C₁˜C₈ alkyl; or
a C₃˜C₈ heterocycloalkyl containing one or more heteroatoms selected from N and O.

2. The compound according to claim 1, wherein:
R₁ is a fluorine atom;
R₂, R₃, and R₃′ are each independently hydrogen or —(X₁)—R₅;
wherein,
X₁ is a straight or branched C₁˜C₈ alkylene, —CH₂(C═O)—, —C(═O)CH₂—, a single bond, —O— or —CO—, and
R₅ is:
a straight or branched C₁˜C₈ alkyl;
a straight or branched C₁˜C₈ alkyl substituted with a C₂˜C₈ dialkylamino;
a straight or branched C₁˜C₈ alkyl substituted with a C₆˜C₂₀ aryl;
a straight or branched C₁˜C₈ alkyl substituted with a halogen-substituted C₆˜C₂₀ aryl;
a straight or branched C₁˜C₈ alkyl substituted with a C₃˜C₈ heterocycloalkyl containing one or more heteroatoms selected from N and O;
a straight or branched C₁˜C₈ alkyl substituted with a C₃˜C₈ heterocycloalkyl which is substituted with a straight or branched C₁˜C₈ alkyl and contains one or more heteroatoms selected from N and O;
a C₃˜C₈ cycloalkyl;
a straight or branched C₁˜C₈ alkanol;
a C₁˜C₈ alkoxy;
a C₂˜C₈ alkenyl;
a C₂˜C₈ alkynyl; hydroxy; carboxy;
a C₆˜C₂₀ aryl;
a C₁˜C₈ acetoxy;
a C₆˜C₂₀ aryl substituted with one or more substituents selected from halogen, cyano, a straight or branched C₁˜C₈ alkyl, CF₃ and a C₁˜C₈ alkoxy;
a C₆˜C₂₀ aryl substituted with a C₃˜C₈ heterocycloalkyl containing one or more heteroatoms selected from N and O;
a C₆˜C₂₀ aryl substituted with a C₃˜C₈ heterocycloalkyl which is substituted with a straight or branched C₁˜C₈ alkyl and contains one or more heteroatoms selected from N and O;
a C₆˜C₂₀ aryl substituted with a straight or branched C₃˜C₈ alkyl substituted with a C₃˜C₈ heterocycloalkyl containing one or more heteroatoms selected from N and O;
a C₆˜C₂₀ aryl substituted with a straight or branched C₁˜C₈ alkyl substituted with a C₃˜C₈ hetetocycloalkyl which is unsubstituted or substituted with a straight or branched C₁˜C₈ alkyl and contains one or more heteroatoms selected from N and O;
a C₆˜C₂₀ aryl substituted with a C₂˜C₈ dialkylamino;
a C₃˜C₈ heterocycloalkyl containing one or more heteroatoms selected from N and O;
a halogen-substituted C₃˜C₈ heterocycloalkyl containing one or more heteroatoms selected from N and O;
phosphonate;
phosphonate which is substituted with a straight or branched C₁˜C₈ alkyl; nitryl; or
a C₁˜C₈ alkylamino,
R₄ is hydrogen, hydroxy, halogen, a C₂˜C₈ dialkylamino or —(X₂)—R₆;
wherein
X₂ is a C₆˜C₂₀ arylene, CO, a single bond or SO₂, and
R₆ is:
a straight or branched C₁˜C₈ alkyl;
a straight or branched C₁˜C₈ alkyl substituted with a halogen-substituted C₆˜C₂₀ aryl;
a C₁˜C₈ alkoxy;
a C₁˜C₂₀ aryl substituted with a C₁˜C₈ alkoxy;
a halogen-substituted C₆˜C₂₀ aryl; or a C₆˜C₂₀ aryl substituted with a halogen-substituted straight or branched C₁˜C₈ alkyl.

3. The compound according to claim 1, wherein the compound has the following Chemical Formula 2: 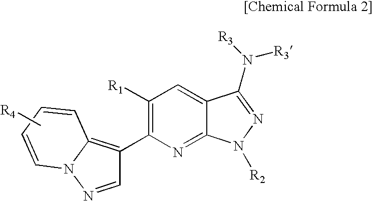
wherein R₁, R₂, R₃, R₃′, and R₄ are as defined as in Chemical Formula 1.

4. The compound according to claim 1, wherein the compound has the following Chemical Formula 3: 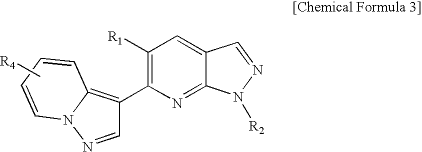
wherein R₁, R₂, and R₄ are as defined as in Chemical Formula 1.

5. A pharmaceutical composition comprising the compound according to claim 1 or a pharmaceutically acceptable salt thereof, and optionally one or more inactive carriers or diluents.

6. A pharmaceutical composition according to claim 5, for treating cancer, diabetes, Alzheimer's disease, CNS disorders or hypertrophic cardiomyopathy.

7. A use of the compound according to claim 1 or a pharmaceutically acceptable salt thereof, in the manufacture of a medicament for inhibiting protein kinase activity in warm-blooded animals.

8. The use according to claim 7, wherein the protein kinase is GSK-3.

9. A method for preparing a compound of Chemical Formula 2 according to claim 3, comprising the steps of:
subjecting a compound of Chemical Formula 4 to the sonogashira reaction to obtain a compound of the following Chemical Formula 5;
reacting the compound of Chemical Formula 5 with a hydrazine compound to obtain a compound of Chemical Formula 6; and
reacting the compound of Chemical Formula 6 with a compound represented by R₂L or with compounds represented by R₃L and R₃′L, to obtain the compound of Chemical Formula 2, 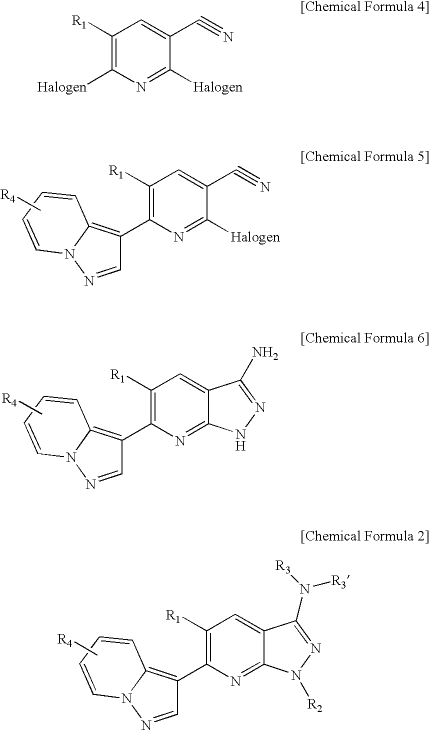
wherein R₁, R₂, R₃, R₃′, and R₄ are as defined as in Chemical Formula 1, and in R₂L, R₃′L and R₃L, L is a leaving group.

10. A method for preparing a compound of Chemical Formula 3 according to claim 4, comprising the steps of:
subjecting a compound of Chemical Formula 4 to a sonogashira reaction to obtain a compound of the following Chemical Formula 5;
reacting the compound of the following Chemical Formula 5 with a hydrazine compound to obtain a compound of the following Chemical Formula 6;
deaminating the compound of the following Chemical Formula 6 to a compound of the following Chemical Formula 7; and
reacting the compound of the following Chemical Formula 7 with a compound represented by R₂L to obtain the compound of Chemical Formula 3, 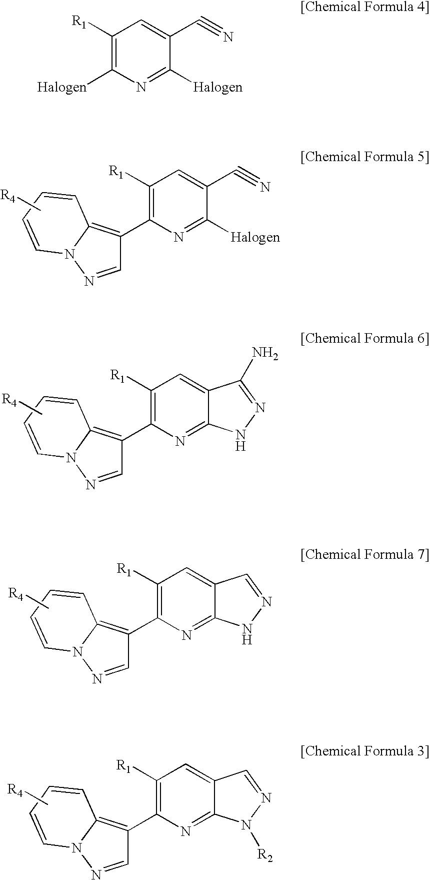
wherein R₁, R₂, and R₄ are as defined as in Chemical Formula 1, and in R₂L, L is a leaving group.
